SEQUENCE LISTING (32 sequences)

SEQ ID NO: 1 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 2 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 3 - protein, 416 aa, Homo sapiens
MEATGISLASQLKVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 4 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 5 - protein, 300 aa, Homo sapiens
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 6 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 7 - protein, 468 aa, Homo sapiens
MAPPPARVHLGAFLAVTPNPGSAASGTEAAAATPSKVWGSSAGRIEPRGGGRGALPTSMGQHGPSARARAGRAPGPRPAREASPRLRVHKTFKFVVVGVLLQVVPSSAATIKLHDQSIGTQQWEHSPLGELCPPGSHRSEHPGACNRCTEGVGYTNASNNLFACLPCTACKSDEEERSPCTTTRNTACQCKPGTFRNDNSAEMCRKCSRGCPRGMVKVKDCTPWSDIECVHKESGNGHNIWVILVVTLVVPLLLVAVLIVCCCIGSGCGGDPKCMDRVCFWRLGLLRGPGAEDNAHNEILSNADSLSTFVSEQQMESQEPADLTGVTVQSPGEAQCLLGPAEAEGSQRRRLLVPANGADPTETLMLFFDKFANIVPFDSWDQLMRQLDLTKNEIDVVRAGTAGPGDALYAMLMKWVNKTGRNASIHTLLDALERMEERHAREKIQDLLVDSGKFIYLEDGTGSAVSLE

SEQ ID NO: 8 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 9 - protein, 263 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKEM

SEQ ID NO: 10 - protein, 430 aa, Homo sapiens
MKPSLLCRPLSCFLMLLPWPLATLTSTTLWQCPPGEEPDLDPGQGTLCRPCPPGTFSAAWGSSPCQPHARCSLWRRLEAQVGMATRDTLCGDCWPGWFGPWGVPRVPCQPCSWAPLGTHGCDEWGRRARRGVEVAAGASSGGETRQPGNGTRAGGPEETAAQYAVIAIVPVFCLMGLLGILVCNLLKRKGYHCTAHKEVGPGPGGGGSGINPAYRTEDANEDTIGVLVRLITEKKENAAALEELLKEYHSKQLVQTSHRPVSKLPPAPPNVPHICPHRHHLHTVQGLASLSGPCCSRCSQKKWPEVLLSPEAVAATTPVPSLLPNPTRVPKAGAKAGRQGEITILSVGRFRVARIPEQRTSSMVSEVKTITEAGPSWGDLPDSPQPGLPPEQQALLGSGGSRTKWLKPPAENKAEENRYVVRLSESNLVI

SEQ ID NO: 11 - protein, 297 aa, Homo sapiens
MDCQENEYWDQWGRCVTCQRCGPGQELSKDCGYGEGGDAYCTACPPRRYKSSWGHHRCQSCITCAVINRVQKVNCTATSNAVCGDCLPRFYRKTRIGGLQDQECIPCTKQTPTSEVQCAFQLSLVEADTPTVPPQEATLVALVSSLLVVFTLAFLGLFFLYCKQFFNRHCQRGGLLQFEADKTAKEESLFPVPPSKETSAESQVSENIFQTQPLNPILEDDCSSTSGFPTQESFTMASCTSESHSHWVHSPIECTELDLQKFSSSASYTGAETLGGNTVESTGDRLELNVPFEVPSP

SEQ ID NO: 12 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 13 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 14 - protein, 922 aa, Homo sapiens
MQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 15 - protein, 201 aa, Homo sapiens
MRLLPLLRTVLWAAFLGSPLRGGSSLRHVVYWNSSNPRLLRGDAVVELGLNDYLDIVCPHYEGPGPPEGPETFALYMVDWPGYESCQAEGPRAYKRWVCSLPFGHVQFSEKIQRFTPFSLGFEFLPGETYYYISVPTPESSGQCLRLQVSVCCKERKSESAHPVGSPGESGTSGWRGGDTPSPLCLLLLLLLLILRLLRIL

SEQ ID NO: 16 - protein, 460 aa, Homo sapiens
MFLATLYFALPLLDLLLSAEVSGGDRLDCVKASDQCLKEQSCSTKYRTLRQCVAGKETNFSLASGLEAKDECRSAMEALKQKSLYNCRCKRGMKKEKNCLRIYWSMYQSLQGNDLLEDSPYEPVNSRLSDIFRVVPFISVEHIPKGNNCLDAAKACNLDDICKKYRSAYITPCTTSVSNDVCNRRKCHKALRQFFDKVPAKHSYGMLFCSCRDIACTERRRQTIVPVCSYEEREKPNCLNLQDSCKTNYICRSRLADFFTNCQPESRSVSSCLKENYADCLLAYSGLIGTVMTPNYIDSSSLSVAPWCDCSNSGNDLEECLKFLNFFKDNTCLKNAIQAFGNGSDVTVWQPAFPVQTTTATTTTALRVKNKPLGPAGSENEIPTHVLPPCANLQAQKLKSNVSGNTHLCISNGNYEKEGLGASSHITTKSMAAPPSCGLSPLLVLVVTALSTLLSLTETS

SEQ ID NO: 17 - protein, 124 aa, Homo sapiens
MPACRLGPLAAALLLSLLLFGFTLVSGTGAEKTGVCPELQADQNCTQECVSDSECADNLKCCSAGCATFCSLPNDKEGSCPQVNINFPQLGLCRDQCQVDSQCPGQMKCCRNGCGKVSCVTPNF

SEQ ID NO: 18 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 19 - protein, 382 aa, Homo sapiens
MRPGTALQAVLLAVLLVGLRAATGRLLSASDLDLRGGQPVCRGGTQRPCYKVIYFHDTSRRLNFEEAKEACRRDGGQLVSIESEDEQKLIEKFIENLLPSDGDFWIGLRRREEKQSNSTACQDLYAWTDGSISQFRNWYVDEPSCGSEVCVVMYHQPSAPAGIGGPYMFQWNDDRCNMKNNFICKYSDEKPAVPSREAEGEETELTTPVLPEETQEEDAKKTFKESREAALNLAYILIPSIPLLLLLVVTTVVCWVWICRKRKREQPDPSTKKQHTIWPSPHQGNSPDLEVYNVIRKQSEADLAETRPDLKNISFRVCSGEATPDDMSCDYDNMAVNPSESGFVTLVSVESGFVTNDIYEFSPDQMGRSKESGWVENEIYGY

SEQ ID NO: 20 - protein, 510 aa, Homo sapiens
MPLSLGAEMWGPEAWLLLLLLLASFTGRCPAGELETSDVVTVVLGQDAKLPCFYRGDSGEQVGQVAWARVDAGEGAQELALLHSKYGLHVSPAYEGRVEQPPPPRNPLDGSVLLRNAVQADEGEYECRVSTFPAGSFQARLRLRVLVPPLPSLNPGPALEEGQGLTLAASCTAEGSPAPSVTWDTEVKGTTSSRSFKHSRSAAVTSEFHLVPSRSMNGQPLTCVVSHPGLLQDQRITHILHVSFLAEASVRGLEDQNLWHIGREGAMLKCLSEGQPPPSYNWTRLDGPLPSGVRVDGDTLGFPPLTTEHSGIYVCHVSNEFSSRDSQVTVDVLDPQEDSGKQVDLVSASVVVVGVIAALLFCLLVVVVVLMSRYHRRKAQQMTQKYEEELTLTRENSIRRLHSHHTDPRSQPEESVGLRAEGHPDSLKDNSSCSVMSEEPEGRSYSTLTTVREIETQTELLSPGSGRAEEEEDQDEGIKQAMNHFVQENGTLRAKPTGNGIYINGRGHLV

SEQ ID NO: 21 - protein, 117 aa, Homo sapiens
MRASSFLIVVVFLIAGTLVLEAAVTGVPVKGQDTVKGRVPFNGQDPVKGQVSVKGQDKVKAQEPVKGPVSTKPGSCPIILIRCAMLNPPNRCLKDTDCPGIKKCCEGSCGMACFVPQ

SEQ ID NO: 22 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 23 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 24 - protein, 700 aa, Homo sapiens
MDLWNLSWFLFLDALLVISGLATPENFDVDGGMDQDIFDINEGLGLDLFEGDIRLDRAQIRNSIIGEKYRWPHTIPYVLEDSLEMNAKGVILNAFERYRLKTCIDFKPWAGETNYISVFKGSGCWSSVGNRRVGKQELSIGANCDRIATVQHEFLHALGFWHEQSRSDRDDYVRIMWDRILSGREHNFNTYSDDISDSLNVPYDYTSVMHYSKTAFQNGTEPTIVTRISDFEDVIGQRMDFSDSDLLKLNQLYNCSSSLSFMDSCSFELENVCGMIQSSGDNADWQRVSQVPRGPESDHSNMGQCQGSGFFMHFDSSSVNVGATAVLESRTLYPKRGFQCLQFYLYNSGSESDQLNIYIREYSADNVDGNLTLVEEIKEIPTGSWQLYHVTLKVTKKFRVVFEGRKGSGASLGGLSIDDINLSETRCPHHIWHIRNFTQFIGSPNGTLYSPPFYSSKGYAFQIYLNLAHVTNAGIYFHLISGANDDQLQWPCPWQQATMTLLDQNPDIRQRMSNQRSITTDPFMTTDNGNYFWDRPSKVGTVALFSNGTQFRRGGGYGTSAFITHERLKSRDFIKGDDVYILLTVEDISHLNSTQIQLTPAPSVQDLCSKTTCKNDGVCTVRDGKAECRCQSGEDWWYMGERCEKRGSTRDTIVIAVSSTVAVFALMLIITLVSVYCTRKKYRERMSSNRPNLTPQNHAF

SEQ ID NO: 25 - protein, 284 aa, Homo sapiens
MGRPLLLPLLLLLQPPAFLQPGGSTGSGPSYLYGVTQPKHLSASMGGSVEIPFSFYYPWELAIVPNVRISWRRGHFHGQSFYSTRPPSIHKDYVNRLFLNWTEGQESGFLRISNLRKEDQSVYFCRVELDTRRSGRQQLQSIKGTKLTITQAVTTTTTWRPSSTTTIAGLRVTESKGHSESWHLSLDTAIRVALAVAVLKTVILGLLCLLLLWWRRRKGLSHRTRPVWNLLYGRSMCGISAELSIGNLDVHISSSKERTRLQAAMSSTSISSKPDSSRAPSSDF

SEQ ID NO: 26 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 27 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 28 - protein, 495 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 29 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 30 - protein, 500 aa, Homo sapiens
MASVAQESAGSQRRLPPRHGALRGLLLLCLWLPSGRAALPPAAPLSELHAQLSGVEQLLEEFRRQLQQERPQEELELELRAGGGPQEDCPGPGSGGYSAMPDAIIRTKDSLAAGASFLRAPAAVRGWRQCVAACCSEPRCSVAVVELPRRPAPPAAVLGCYLFNCTARGRNVCKFALHSGYSSYSLSRAPDGAALATARASPRQEKDAPPLSKAGQDVVLHLPTDGVVLDGRESTDDHAIVQYEWALLQGDPSVDMKVPQSGTLKLSHLQEGTYTFQLTVTDTAGQRSSDNVSVTVLRAAYSTGGCLHTCSRYHFFCDDGCCIDITLACDGVQQCPDGSDEDFCQNLGLDRKMVTHTAASPALPRTTGPSEDAGGDSLVEKSQKATAPNKPPALSNTEKRNHSAFWGPESQIIPVMPDSSSSGKNRKEESYIFESKGDGGGGEHPAPETGAVLPLALGLAITALLLLMVACRLRLVKQKLKKARPITSEESDYLINGMYL

SEQ ID NO: 31 - protein, 424 aa, Homo sapiens
MRAPGCGRLVLPLLLLAAAALAEGDAKGLKEGETPGNFMEDEQWLSSISQYSGKIKHWNRFRDEVEDDYIKSWEDNQQGDEALDTTKDPCQKVKCSRHKVCIAQGYQRAMCISRKKLEHRIKQPTVKLHGNKDSICKPCHMAQLASVCGSDGHTYSSVCKLEQQACLSSKQLAVRCEGPCPCPTEQAATSTADGKPETCTGQDLADLGDRLRDWFQLLHENSKQNGSASSVAGPASGLDKSLGASCKDSIGWMFSKLDTSADLFLDQTELAAINLDKYEVCIRPFFNSCDTYKDGRVSTAEWCFCFWREKPPCLAELERIQIQEAAKKKPGIFIPSCDEDGYYRKMQCDQSSGDCWCVDQLGLELTGTRTHGSPDCDDIVGFSGDFGSGVGWEDEEEKETEEAGEEAEEEEGEAGEADDGGYIW

SEQ ID NO: 32 - protein, 750 aa, Homo sapiens
MEDYPDPQSANHMNSSLLSLYRKGNPDSVSNTPEMEQRETTSSTPRISSKTGSIPLKTPAKDSEGGWFIDKTPSVKKDSFFLDLSCEKSNPKKPITEIQDSKDSSLLESDMKRKGKLKNKGSKRKKEDLQEVDGEIEAVLQKKAKARGLEFQISNVKFEDVGGNDMTLKEVCKMLIHMRHPEVYHHLGVVPPRGVLLHGPPGCGKTLLAHAIAGELDLPILKVAAPEIVSGVSGESEQKLRELFEQAVSNAPCIIFIDEIDAITPKREVASKDMERRIVAQLLTCMDDLNNVAATARVLVIGATNRPDSLDPALRRAGRFDREICLGIPDEASRERILQTLCRKLRLPQAFDFCHLAHLTPGFVGADLMALCREAAMCAVNRVLMKLQEQQKKNPEMEDLPSKGVQEERLGTEPTSETQDELQRLLGLLRDQDPLSEEQMQGLCIELNDFIVALSSVQPSAKREGFVTVPNVTWADIGALEDIREELTMAILAPVRNPDQFKALGLVTPAGVLLAGPPGCGKTLLAKAVANESGLNFISVKGPELLNMYVGESERAVRQVFQRAKNSAPCVIFFDEVDALCPRRSDRETGASVRVVNQLLTEMDGLEARQQVFIMAATNRPDIIDPAILRPGRLDKTLFVGLPPPADRLAILKTITKNGTKPPLDADVNLEAIAGDLRCDCYTGADLSALVREASICALRQEMARQKSGNEKGELKVSHKHFEEAFKKVRSSISKKDQIMYERLQESLSR

(19) United States
(12) Patent Application Publication
(10) Pub. No.: US 2025/0321233 A1
(43) Pub. Date: Oct. 16, 2025

(54) PRECISION MEDICINE FOR TREATMENT OF KIDNEY FUNCTION DECLINE

(71) Applicant: Joslin Diabetes Center, Inc., Boston, MA (US)

(72) Inventor: Andrzej Krolewski, Needham, MA (US)

(21) Appl. No.: 18/956,971

(22) Filed: Nov. 22, 2024

Related U.S. Application Data
(63) Continuation of application No. PCT/US2023/067428, filed on May 24, 2023.
(60) Provisional application No. 63/365,262, filed on May 24, 2022.

(51) Int. Cl. G01N 33/68

(52) U.S. Cl. CPC G01N 33/68; G01N 2333/4703; G01N 2333/715; G01N 2800/347; G01N 2800/52

(57) ABSTRACT

The invention provides methods and compositions for identifying a subject who will responds to a reno-protective agent for treating or preventing progressive kidney function decline based on the level of a renal associated protein.

### CROSS-REFERENCE TO RELATED APPLICATIONS

This application is a continuation of PCT/US2023/067428, filed on May 24, 2023, which claims priority to U.S. Provisional Application No. 63/365,262, filed on May 24, 2022, the entirety of which is incorporated by reference herein.

### SEQUENCE LISTING

The instant application contains a Sequence Listing which has been submitted in XML format and is hereby incorporated by reference in its entirety. Said XML copy, created on May 24, 2023, is named J103021_1100WO_SL_ST26 and is 40.5 kilobytes in size.

### BACKGROUND

More than 1 in 7 of US adults are estimated to have chronic kidney disease (CKD) according to the Center for Disease Control. Kidney diseases are a leading cause of death in the United States. When people develops CKD, their kidneys become damaged over time and do not filter blood efficiently, toxic waste and extra fluid accumulate in the body and may lead to high blood pressure, heart disease, stroke, and early death. If left untreated, CKD can progress to kidney failure and early cardiovascular disease. Patients diagnosed with stage 3-5 CKD of have poor prognosis. For example, elderly patients with stage 5 CKD have a life expectancy of a few months (Reindl-Schwaighofer et al., PLoS ONE, 2017, 12(7): e0181345). The personal impact, as well as the cost, of kidney disease on society is high.

Given the progressive, detrimental effect kidney disease can have on a patient's life, treatment of kidney function decline should be selected in a way that will maximize the efficacy for the patient. Being able to understand how a patient will respond to a given therapy is, however, a challenge in the field.

### SUMMARY OF THE INVENTION

Understanding how a patient will respond to kidney disease therapy is important in helping the patient battle this devastating disease. The present disclosure presents protein markers for predicting a patient's response and determining effective medicine for preventing and/or treating progressive kidney function decline.

One aspect of the present disclosure provides a method for determining whether a human subject will respond to a reno-protective agent for the treatment or prevention of progressive kidney function decline, said method comprising detecting the level of a renal associated protein in a biological sample from a human subject having or at risk of having progressive kidney function decline. In some embodiments, the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof. The method further comprises comparing the level of the renal associated protein with a responder control level. In some embodiments, the human subject is a responder to the reno-protective agent if the level of the renal associated protein is equal to or higher than the responder control level, and wherein the human subject is not a responder to the reno-protective agent if the level of the renal associated protein is less than the responder control level.

Another aspect of the present disclosure provides a method for determining whether a human subject will respond to a reno-protective agent for the treatment or prevention of progressive kidney function decline, said method comprising detecting the level of a renal associated protein in a biological sample from a human subject having or at risk of having progressive kidney function decline. In some embodiments, the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof. The method further comprises comparing the level of the renal associated protein with a non-responder control level. In some embodiments, the human subject is a non-responder to the reno-protective agent if the level of the renal associated protein is equal to or higher than the non-responder control level, and wherein the human subject is not a non-responder to the reno-protective agent if the level of the renal associated protein is less than the non-responder control level.

In one specific embodiment, the reno-protective agent agent is fenofibrate. In another specific embodiment, the reno-protective agent agent is baricitinib. In yet another specific embodiment, the reno-protective agent agent is an SGLT2 inhibitor. In yet another specific embodiment, the reno-protective agent is a GLP-1/GIP agonist.

In some embodiments, the protein level is determined by an assay selected from the group consisting of an immunoassay, a mass spectrometry analysis, a Slow Off-rate Modified Aptamer (SOMA) scan platform analysis, liquid chromatography (LC) fractionation, Mesoscale platform, electrochemiluminescence detection, or an OLINK Proximity Extension Assay based proteomic platform analysis.

In some embodiments, the kidney function decline is progression from normal kidney function to chronic kidney disease.

In some embodiments, the kidney function decline is progression from chronic kidney disease to end stage kidney disease (ESKD).

In some embodiments, the human subject has type 1 diabetes (T1D). In some other embodiments, the human subject has type 2 diabetes (T2D). In yet some other embodiments, the human subject has diabetic kidney disease

In some embodiments, the human subject has cystinosis, glomerulonephritis, polycystic kidney disease, or IgA nephropathy.

In some further embodiments, the human subject is in early progressive renal decline.

In some other further embodiments, the human subject is in late progressive renal decline.

In another aspect of the present disclosure, further provided is a method for treating or preventing progressive kidney function decline in a human subject, the method comprising determining whether the human subject will respond to a reno-protective agent for the treatment or prevention of progressive kidney function decline, comprising the steps of: detecting the level of a renal associated protein in a biological sample from the human subject. In some embodiments, the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof. The method further comprises comparing the level of the renal associated protein with a responder control level. In some embodiments, the human subject is a responder to the reno-protective agent if the level of the renal associated protein is equal to or higher than a responder control level. In some other embodiments, the human subject is not a responder to the reno-protective agent if the level of the renal associated protein is less than the responder control level. The method further comprise administering the reno-protective agent to the responder, such that the progressive kidney function decline is treated or prevented.

In one specific embodiment, the reno-protective agent agent is fenofibrate. In another specific embodiment, the reno-protective agent agent is baricitinib. In yet another specific embodiment, the reno-protective agent agent is an SGLT2 inhibitor. In yet another specific embodiment, the reno-protective agent is a GLP-1/GIP agonist.

In another specific aspect, the present disclosure provides a method for determining whether a human subject will respond to fenofibrate for the treatment or prevention of progressive kidney function decline, said method comprising detecting the level of a renal associated protein in a biological sample from the human subject, wherein the renal associated protein is EFNA4, DLL1, or a combination thereof, and comparing the level of the renal associated protein with a responder control level. In some embodiments, the human subject is a responder to fenofibrate if the level of the renal associated protein is equal to or higher than a responder control level. In some other embodiments, the human subject is not a responder to fenofibrate if the level of the renal associated protein is less than the responder control level.

In some embodiments, the method further comprises administering an effective amount of fenofibrate to the responder such that the progressive kidney function decline is treated.

In another specific aspect, the present disclosure provides a method for determining whether a human subject will respond to baricitinib for the treatment or prevention of progressive kidney function decline, said method comprising detecting the level of a renal associated protein in a biological sample from the human subject, wherein the renal associated protein is TNF-RSF7, IL-1RT1, or a combination thereof, and comparing the level of the renal associated protein with a responder control level. In some embodiments, the human subject is a responder to baricitinib if the level of the renal associated protein is equal to or higher than a responder control level. In some other embodiments, the human subject is not a responder to baricitinib if the level of the renal associated protein is less than the responder control level.

In some embodiments, the method further comprises administering an effective amount of baricitinib to the responder such that the progressive kidney function decline is treated.

In yet another specific aspect, the present disclosure provides a method for determining whether a human subject will respond to SGL2 for the treatment or prevention of progressive kidney function decline, said method comprising detecting the level of a renal associated protein in a biological sample from the human subject, and comparing the level of the renal associated protein with a responder control level. In some embodiments, the human subject is a responder to SGL2 if the level of the renal associated protein is equal to or higher than a responder control level. In some other embodiments, the human subject is not a responder to SGL2 if the level of the renal associated protein is less than the responder control level.

In some embodiments, the method further comprises administering an effective amount of SGL2 to the responder such that the progressive kidney function decline is treated.

In some embodiments, the protein level is determined by an assay selected from the group consisting of an immunoassay, a mass spectrometry analysis, a Slow Off-rate Modified Aptamer (SOMA) scan platform analysis, liquid chromatography (LC) fractionation, Mesoscale platform, electrochemiluminescence detection, or an OLINK Proximity Extension Assay based proteomic platform analysis.

In some embodiments, the kidney function decline is progression from normal kidney function to chronic kidney disease.

In some embodiments, the kidney function decline is progression from chronic kidney disease to ESKD.

In some embodiments, the human subject has type 1 diabetes (T1D). In some other embodiments, the human subject has type 2 diabetes (T2D).

In some embodiments, the human subject has diabetic kidney disease.

In one embodiment, the human subject has chronic kidney disease (CKD).

In some embodiments, the human subject has cystinosis, glomerulonephritis, polycystic kidney disease, or IgA nephropathy.

In some embodiments, the kidney disease is early progressive renal decline. In some other embodiments, the kidney disease is late progressive renal decline.

### BRIEF DESCRIPTION OF THE DRAWINGS

FIG. 1. FIG. 1A is graph showing the estimated glomerular filtration rate slope in Joslin cohort who have varied KIM1 protein levels. FIG. 1B is a graph showing the TNF-RSF1A protein level in non-ESKD patients and ESDK patients of the Joslin cohort.

FIG. 2 is a graph showing the odds ratio and p-value demonstrating the association of baseline levels of markers with ESKD risk in univariable logistic model in Pima cohort.

FIG. 3. FIG. 3A is a graph showing association of the response to fenofibrate treatment and the levels of EFNA4 and DLL1 in ACCORD cohort. FIG. 3B is a graph showing the change (%) of TNF-RSF7 and IL-1RT1 protein level in placebo or 4 mg-baricitinib-treated group.

### DETAILED DESCRIPTION

### I. Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

As used herein, the term “kidney disease” refers to a conditions or disease characterized by a decrease in kidney function compared to a healthy patient. Kidney disease may be characterized as nephrotic syndrome or renal insufficiency. A characteristic of kidney disease is a progressive kidney function decline, resulting in chronic kidney disease and eventually in end stage kidney disease (ESKD). Examples of kidney disease include, but are not limited to, diabetic kidney disease, cystinosis, glomerulonephritis, polycystic kidney disease, or IgA nephropathy.

As used herein, the term “end stage kidney disease” or “ESKD”, refers to the last stage of chronic kidney disease (CKD) which occurs when a person's kidneys can no longer support their body's needs.

A subject “at risk of having or at risk of developing progressive kidney function decline” is a subject who may, in certain embodiments, have a disorder associated with kidney disease, e.g., a person who has hypertension or obesity.

The term “reno-protective agent”, as used herein, refers to a therapeutic agent that can improve, e.g., restore kidney function, maintain kidney function (e.g., by preventing further decline), or reduce progression of kidney decline, e.g., reduce the time to ESKD, when administered to a subject in need thereof.

The term “renal associated protein”, as used herein, generally refers to a peptide or a polypeptide whose expression predicts or indicates whether a reno-protective agent will provide therapeutic benefit for the treatment or prevention of progressive kidney function decline or detrimental decline of kidney function. Renal associated proteins described herein include TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, and NVL1.

The term “level” or “amount” of a renal protective protein, as used herein, refers to the measurable quantity of the protein. The amount may be either (a) an absolute amount as measured in molecules, moles or weight per unit volume or cells or (b) a relative amount, e.g., measured by densitometric analysis.

As used herein, the term “responder” refers to a subject who has positive response to a reno-protective agent when administered to treat or prevent progressive kidney function decline. A response to a reno-protective agent for treating or preventing progressive kidney function decline can include, but is not limited to, alleviation of symptoms associated with kidney decline. A responder also includes a person who does not develop kidney function decline despite being at risk thereof, when administered the reno-protective agent. The treatment response can be evaluated or assessed using methods known in the art.

As used herein, the term “reference” level can also be referred to as a “control” level. A reference level can be generated from a sample taken from a normal individual or from an individual known to have a predisposition to progressive kidney function decline, or from an individual known to have progressive kidney function decline. The reference level, or plurality of reference levels, can be used to establish threshold values for the levels of, for example, specific renal protective protein markers in a sample. A “reference” level includes levels generated from one or more subjects having a predisposition to progressive kidney function decline, levels generated from one or more subjects having progressive kidney function decline, or levels generated from one or more normal, healthy subjects who do not have progressive kidney function decline.

A reference level can be in the form of a threshold value or series of threshold values. For example, a single threshold value can be determined by averaging the values of a series of levels of a single biomarker from subjects having no predisposition to progressive kidney function decline. Similarly, a single threshold value can be determined by averaging the values of a series of levels of a single biomarker from subjects having a predisposition to progressive kidney function decline. Thus, a threshold value can have a single value or a plurality of values, each value representing a level of a specific biomarker, detected in a plasma sample, e.g., of an individual, or multiple individuals, having a predisposition progressive kidney function decline.

As used herein, the term “responder control level” refers to an accepted or pre-determined level of a renal associated protein that predicts a positive response to a reno-protective agent for treating or preventing progressive kidney function decline in a subject. A subject whose renal protective protein level is less than the responder control level is considered a non-responder to the therapeutic. In certain embodiments, the level is one standard deviation or more below the responder control level.

The term “non-responder control level” refers to an accepted or pre-determined level of a renal associated protein that predicts a lack of response to the reno-protective agent for treating or preventing progressive kidney function decline in a subject. In one embodiment, a human subject is a non-responder to a reno-protective agent for treating or preventing progressive kidney function decline if the level of the renal associated protein is equal or greater than the non-responder control level.

As used herein, the term “estimated Glomerular Filtration Rate” or “eGFR”, refers to a means for estimating kidney function. In one embodiment, eGFR may be determined based on a measurement of serum creatinine levels. In another embodiment, eGFR may be determined based on a measurement of serum cystatin C levels. eGFR can be determined using the CKD-EPI creatinine equation, as described, for example, in Levey et al. (Ann Intern Med 150(9): 604-61221 (2009)).

The term “sample” or “biological sample” as used herein refers to cells or tissue obtained from a subject. The source of the tissue or cell sample may be solid tissue (as from a fresh, frozen and/or preserved organ or tissue sample or biopsy or aspirate); whole blood or any blood constituents; or bodily fluids, such as serum, plasma, urine, saliva, sweat or synovial fluid. In one embodiment, the sample is a plasma sample obtained from a human subject. In another embodiment, the sample is a urine sample obtained from a human subject.

The term “subject” or “patient”, as used interchangeably herein, refers to either a mammal, including mice, humans, and primates. Preferably, the subject is a human subject.

As used herein, the term “kidney function decline” refers to progressive kidney function decline and is measured as a slope (change) in the estimated Glomerular Filtration Rate (eGFR) per year. For example, in one embodiment, kidney function decline is an eGFR change of at least <−3 ml/min/year. In one embodiment, an eGFR change of between −5 ml/min/year and −3 ml/min/year indicates slow or moderate kidney decline. In another embodiment, an eGFR change of between −80 ml/min/year and −5 ml/min/year indicates fast kidney decline.

As used herein, the term “treatment” or “treating” refers to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

As used herein, the term “prevent” or “preventing” refers to inhibiting a disease, disorder or condition in a subject, e.g., impeding its progress; and/or relieving the disease, disorder or condition, e.g., causing regression of the disease, disorder and/or condition, in a subject having a disease or who may be predisposed to the disease, disorder and/or condition but has not yet been diagnosed as having it. In one embodiment, the methods disclosed herein prevent kidney function decline, particularly progressive kidney function decline.

The term “therapeutically effective amount” refers to an amount effective, at dosages and for periods of time necessary, to achieve the desired therapeutic result or prophylactic result. In one embodiment, the methods disclosed herein comprising administering a therapeutically effective amount of a reno-protective agent.

### II. Precision Methods and Compositions Comprising Renal Associated Proteins

The present disclosure is based, at least in part, on the identification of certain renal associated proteins whose levels can be used to predict whether a human subject will respond to a reno-protective agent for preventing or treating progressive kidney function decline. A renal associated protein may be used to determine whether a subject having progressive kidney function decline will be a responder or a non-responder to a reno-protective agent for treating or preventing progressive kidney function decline. The determination is based on the level of the renal associated protein from a biological sample of the subject, where the level is compared to a control level. Accordingly, in one embodiment, a renal associated protein useful in the present disclosure, is any molecule (or combination of molecules) the expression of which is regulated (up or down) in a subject who is a responder of a reno-protective agent for treating or preventing progressive kidney function decline, when compared to a non-responder of the said reno-protective agent. In one embodiment, selected sets of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more of the renal associated proteins as disclosed herein can be used as markers for determining the prognostics for whether a patient is a responder of a reno-protective agent for treating or preventing progressive kidney function decline.

### Renal Associated Proteins

The renal associated proteins which can be measured in a sample from a subject include TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, and NVL1. A description of each of the renal protective proteins identified herein that can be used in the methods and compositions of the invention is provided as follows. Notably, the proteins can be used individually or in combination to determine which reno-protective agent will be effective for the subject in need thereof.

### TNF-RSF1A

Human TNF-RSF1A, also known as TNF receptor superfamily member 1A, TNFRSF1A, FPF, p55, p60, TBP1, TNF-R, TNFAR, TNFR1, p55-R, CD120a, TNFR55, TNFR60, TNF-R-I and TNF-R55, is a member of the TNF receptor superfamily of proteins. TNF-RSF1A is found in membrane-bound and soluble forms that interact with membrane-bound and soluble forms, respectively, of its ligand, tumor necrosis factor alpha. Binding of membrane-bound tumor necrosis factor alpha to the membrane-bound TNF-RSF1A induces trimerization and activation, which plays a role in cell survival, apoptosis, and inflammation. Proteolytic processing of TNF-RSF1A results in release of the soluble form of it, which can interact with free tumor necrosis factor alpha to inhibit inflammation.

The amino acid sequence of human TNF-RSF1A (NP_001056.1, isoform 1 precursor) is provided below:

(SEQ ID NO: 1)
MGLSTVPDLLLPLVLLELLVGIYPSGVIGLVPHLGDREKRDSVCPQGKY
IHPQNNSICCTKCHKGTYLYNDCPGPGQDTDCRECESGSFTASENHLRH
CLSCSKCRKEMGQVEISSCTVDRDTVCGCRKNQYRHYWSENLFQCFNCS
LCLNGTVHLSCQEKQNTVCTCHAGFFLRENECVSCSNCKKSLECTKLCL
PQIENVKGTEDSGTTVLLPLVIFFGLCLLSLLFIGLMYRYQRWKSKLYS
IVCGKSTPEKEGELEGTTTKPLAPNPSFSPTPGFTPTLGFSPVPSSTFT
SSSTYTPGDCPNFAAPRREVAPPYQGADPILATALASDPIPNPLQKWED
SAHKPQSLDTDDPATLYAVVENVPPLRWKEFVRRLGLSDHEIDRLELQN
GRCLREAQYSMLATWRRRTPRREATLELLGRVLRDMDLLGCLEDIEEAL
CGPAALPPAPSLLR

TNF-RSF1A sequences can also be found in publicly available databases, for example, GenBank, OMIM, and UniProt (P19438).

### TNF-RSF1B

Human TNF-RSF1B, also known as TNF receptor superfamily member 1B, TNFRSF1B, p75, TBPII, TNFBR, TNFR2, CD120b, TNFR1B, TNFR80, TNF-R75, p75TNFR, and TNF-R-II. TNF-RSF1B is a member of the TNF receptor superfamily of proteins. TNF-RSF1B and TNF-receptor 1 form a heterocomplex that mediates the recruitment of two anti-apoptotic proteins, c-IAP1 and c-IAP2, which possess E3 ubiquitin ligase activity. TNF-RSF1B is associated with kidney disease. For example, soluble TNF-RSF1B is associated with progressive diabetic kidney disease in patients with type 2 diabetes mellitus (Wu et al., PLoS ONE, 2022,17(4):e0266854).

The amino acid sequence of human TNF-RSF1R (NP_001057.1) is provided below

(SEQ ID NO: 2)
MAPVAVWAALAVGLELWAAAHALPAQVAFTPYAPEPGSTCRLREYYDQT
AQMCCSKCSPGQHAKVFCTKTSDTVCDSCEDSTYTQLWNWVPECLSCGS
RCSSDQVETQACTREQNRICTCRPGWYCALSKQEGCRLCAPLRKCRPGF
GVARPGTETSDVVCKPCAPGTFSNTTSSTDICRPHQICNVVAIPGNASM
DAVCTSTSPTRSMAPGAVHLPQPVSTRSQHTQPTPEPSTAPSTSFLLPM
GPSPPAEGSTGDFALPVGLIVGVTALGLLIIGVVNCVIMTQVKKKPLCL
QREAKVPHLPADKARGTQGPEQQHLLITAPSSSSSSLESSASALDRRAP
TRNQPQAPGVEASGAGEARASTGSSDSSPGGHGTQVNVTCIVNVCSSSD
HSSQCSSQASSTMGDTDSSPSESPKDEQVPFSKEECAFRSQLETPETLL
GSTEEKPLPLGVPDAGMKPS

TNF-RSF1B sequences can also be found in publicly available databases, for example, GenBank, OMIM, and UniProt (P20333).

### TNF-RSF3

Human TNF-RSF3, also known as lymphotoxin beta receptor, LTBR, TNFCR, TNFR3, D12S370, TNFR-RP, TNFRSF3; TNFR2-RP, LT-BETA-R, and TNF-R-III. TNF-RSF3 is a member of the TNF receptor superfamily of proteins. The major ligands of TNF-RSF3 include lymphotoxin alpha/beta and tumor necrosis factor ligand superfamily member 14.

The amino acid sequence of human TNF-RSF3 (NP_001257916.1) is provided below:

(SEQ ID NO: 3)
MEATGISLASQLKVPPYASENQTCRDQEKEYYEPQHRICCSRCPPGTYV
SAKCSRIRDTVCATCAENSYNEHWNYLTICQLCRPCDPVMGLEEIAPCT
SKRKTQCRCQPGMFCAAWALECTHCELLSDCPPGTEAELKDEVGKGNNH
CVPCKAGHFQNTSSPSARCQPHTRCENQGLVEAAPGTAQSDTTCKNPLE
PLPPEMSGTMLMLAVLLPLAFFLLLATVFSCIWKSHPSLCRKLGSLLKR
RPQGEGPNPVAGSWEPPKAHPYFPDLVQPLLPISGDVSPVSTGLPAAPV
LEAGVPQQQSPLDLTREPQLEPGEQSQVAHGTNGIHVTGGSMTITGNIY
IYNGPVLGGPPGPGDLPATPEPPYPIPEEGDPGPPGLSTPHQEDGKAWH
LAETEHCGATPSNRGPRNQFITHD

TNF-RSF3 sequences can also be found in publicly available databases, for example, GenBank, OMIM, and UniProt (P36941).

### TNF-RSF4

Human TNF-RSF4, also known as TNF receptor superfamily member 4, TNFRSF4, OX40, ACT35, CD134, IMD16 and TXGP1L. TNF-RSF4 is a member of the TNF receptor superfamily of proteins, has been shown to activate NF-kappaB through its interaction with adaptor proteins TRAF2 and TRAF5. TNF-RSF4 is reported to have highly similar activity in T cell activation despite its unique cytoplasmic domain structure and signaling pathway (Schreiber et al., J Immunol, 2012, 189:3311-3318).

The amino acid sequence of human TNF-RSF4 (NP_003318.1) is provided below:

(SEQ ID NO: 4)
MCVGARRLGRGPCAALLLLGLGLSTVTGLHCVGDTYPSNDRCCHECRP
GNGMVSRCSRSQNTVCRPCGPGFYNDVVSSKPCKPCTWCNLRSGSERK
QLCTATQDTVCRCRAGTQPLDSYKPGVDCAPCPPGHFSPGDNQACKPW
TNCTLAGKHTLQPASNSSDAICEDRDPPATQPQETQGPPARPITVQPT
EAWPRTSQGPSTRPVEVPGGRAVAAILGLGLVLGLLGPLAILLALYLL
RRDQRLPPDAHKPPGGGSFRTPIQEEQADAHSTLAKI

TNF-RSF4 sequences can also be found in publicly available databases, for example, GenBank, OMIM, and UniProt (P43489).

### TNF-RSF6B

Human TNF-RSF6B, also known as TNF receptor superfamily member 6b, TNFRSF6B, M68; TR6; DCR3; M68E, and DJ583P15.1.1. TNF-RSF6B is a member of the TNF receptor superfamily of proteins, but lacks transmembrane and cytoplasmic domains in its sequence. TNF-RSF6B is postulated to play a regulatory role in suppressing FasL- and LIGHT-mediated cell death. Serum TNF-RSF6B level is markedly elevated in patients with chronic kidney disease (Tseng et al., Modern Pathology, 2013, 26: 984-994; Chen et al., J Immunol Methods, 2004, 285:63-70).

The amino acid sequence of human TNF-RSF6B (NP_003814.1) is provided below:

(SEQ ID NO: 5)
MRALEGPGLSLLCLVLALPALLPVPAVRGVAETPTYPWRDAETGERLV
CAQCPPGTFVQRPCRRDSPTTCGPCPPRHYTQFWNYLERCRYCNVLCG
EREEEARACHATHNRACRCRTGFFAHAGFCLEHASCPPGAGVIAPGTP
SQNTQCQPCPPGTFSASSSSSEQCQPHRNCTALGLALNVPGSSSHDTL
CTSCTGFPLSTRVPGAEECERAVIDFVAFQDISIKRLQRLLQALEAPE
GWGPTPRAGRAALQLKLRRRLTELLGAQDGALLVRLLQALRVARMPGL
ERSVRERFLPVH

TNF-RSF6B sequences can also be found in publicly available databases, for example, GenBank, OMIM, and UniProt (095407).

### TNF-RSF7

Human TNF-RSF7, also known as CD27, Tumor Necrosis Factor Receptor Superfamily Member 7, T14, S152, Tp55, TNFRSF7, S152 and LPFS2, is a member of the TNF receptor superfamily of proteins.

The amino acid sequence of human TNF-RSF7 NP 001233.2 is provided below:

(SEQ ID NO: 6)
MARPHPWWLCVLGTLVGLSATPAPKSCPERHYWAQGKLCCQMCEPGTF
LVKDCDQHRKAAQCDPCIPGVSFSPDHHTRPHCESCRHCNSGLLVRNC
TITANAECACRNGWQCRDKECTECDPLPNPSLTARSSQALSPHPQPTH
LPYVSEMLEARTAGHMQTLADFRQLPARTLSTHWPPQRSLCSSDFIRI
LVIFSGMFLVFTLAGALFLHQRRKYRSNKGESPVEPAEPCHYSCPREE
EGSTIPIQEDYRKPEPACSP

TNF-RSF7 sequences can also be found in publicly available databases, for example, GenBank, OMIM, and UniProt (P26842).

### TNF-RSF10A

Human TNF-RSF10A, also known as Tumor Necrosis Factor Receptor Superfamily Member 10A, TNFRSF10A, DR4, APO2, CD261, TRAILR1 and TRAILR-1, is a member of the TNF receptor superfamily of proteins. It is a cell surface receptor that bind to TRAIL 3 and mediate the extrinsic pathway of apoptosis. Studies with FADD-deficient mice suggested that FADD, a death domain containing adaptor protein, is required for the apoptosis mediated by this protein. The up-regulation of TNF-RSF10A expression usually occurs at the transcriptional level via transcriptional factors AP-1, TP53, or NF-κB (Li et al., Gene Regulation, 2015, 290(17) P11108-11118).

The amino acid sequence of human TNF-RSF10A (NP_003835.3) is provided below:

(SEQ ID NO: 7)
MAPPPARVHLGAFLAVTPNPGSAASGTEAAAATPSKVWGSSAGRIEPR
GGGRGALPTSMGQHGPSARARAGRAPGPRPAREASPRLRVHKTFKFVV
VGVLLQVVPSSAATIKLHDQSIGTQQWEHSPLGELCPPGSHRSEHPGA
CNRCTEGVGYTNASNNLFACLPCTACKSDEEERSPCTTTRNTACQCKP
GTFRNDNSAEMCRKCSRGCPRGMVKVKDCTPWSDIECVHKESGNGHNI
WVILVVTLVVPLLLVAVLIVCCCIGSGCGGDPKCMDRVCFWRLGLLRG
PGAEDNAHNEILSNADSLSTFVSEQQMESQEPADLTGVTVQSPGEAQC
LLGPAEAEGSQRRRLLVPANGADPTETLMLFFDKFANIVPFDSWDQLM
RQLDLTKNEIDVVRAGTAGPGDALYAMLMKWVNKTGRNASIHTLLDAL
ERMEERHAREKIQDLLVDSGKFIYLEDGTGSAVSLE

TNF-RSF10A sequences can also be found in publicly available databases, for example, GenBank, OMIM, and UniProt (000220).

### TNF-RSF10B

Human TNF-RSF10B, also known as Tumor Necrosis Factor Receptor Superfamily Member 10B, TNFRSF10B, DR5, CD262, KILLER, TRICK2, TRICKB, ZTNFR9, TRAILR2, TRICK2A, TRICK2B, TRAIL-R2 and KILLER/DR5, is a member of the TNF receptor superfamily of proteins, and contains an intracellular death domain. Studies with FADD-deficient mice suggested that FADD, a death domain containing adaptor protein, is required for the apoptosis mediated by TNF-RSF10B (MacFarlane et al., J. Biol. Chem., 1997, 272 (41): 25417-20; Chaudhary et al., Immunity, 1997, 7 (6): 821-30).

The amino acid sequence of human TNF-RSF10B (NP_003833.4) is provided below:

(SEQ ID NO: 8)
MEQRGQNAPAASGARKRHGPGPREARGARPGPRVPKTLVLVVAAVLLL
VSAESALITQQDLAPQQRAAPQQKRSSPSEGLCPPGHHISEDGRDCIS
CKYGQDYSTHWNDLLFCLRCTRCDSGEVELSPCTTTRNTVCQCEEGTF
REEDSPEMCRKCRTGCPRGMVKVGDCTPWSDIECVHKESGTKHSGEVP
AVEETVTSSPGTPASPCSLSGIIIGVTVAAVVLIVAVFVCKSLLWKKV
LPYLKGICSGGGGDPERVDRSSQRPGAEDNVLNEIVSILQPTQVPEQE
MEVQEPAEPTGVNMLSPGESEHLLEPAEAERSQRRRLLVPANEGDPTE
TLRQCFDDFADLVPFDSWEPLMRKLGLMDNEIKVAKAEAAGHRDTLYT
MLIKWVNKTGRDASVHTLLDALETLGERLAKQKIEDHLLSSGKFMYLE
GNADSAMS

TNF-RSF10B sequences can also be found in publicly available databases, for example, GenBank, OMIM, and UniProt (014763).

### TNF-RSF11A

Human TNF-RSF11A, also known as Tumor Necrosis Factor Receptor Superfamily Member 11A, TNFRSF11A, FEO, OFE, ODFR, OSTS, PDB2, RANK, CD265, OPTB7, TRANCER, LOH18CR1, and TRANCE-R, is a member of the TNF receptor superfamily of proteins. TNF-RSF11A is a mediator for osteoclast and lymph node development.

The amino acid sequence of human TNF-RSF11A (NP_001257878.1) is provided below:

(SEQ ID NO: 9)
MAPRARRRRPLFALLLLCALLARLQVALQIAPPCTSEKHYEHLGRCCN
KCEPGKYMSSKCTTTSDSVCLPCGPDEYLDSWNEEDKCLLHKVCDTGK
ALVAVVAGNSTTPRRCACTAGYHWSQDCECCRRNTECAPGLGAQHPLQ
LNKDTVCKPCLAGYFSDAFSSTDKCRPWTNCTFLGKRVEHHGTEKSDA
VCSSSLPARKPPNEPHVYLPGLIILLLFASVALVAAIIFGVCYRKKGK
ALTANLWHWINEACGRLSGDKEM

TNF-RSF11A sequences can also be found in publicly available databases, for example, GenBank, OMIM, and UniProt (Q9Y6Q6).

### TNF-RSF19L

Human TNF-RSF19L, also known as RELT TNF receptor, RELT, AI3C and TRLT, is a member of the TNF receptor superfamily of proteins. TNF-RSF19L is especially abundant in hematologic tissues. It has been shown to activate the NF-kappaB pathway and selectively bind TNF receptor-associated factor 1 (TRAF1). It is capable of stimulating T-cell proliferation in the presence of CD3 signaling, which suggests its regulatory role in immune response.

The amino acid sequence of human TNF-RSF19L (NP_116260.2) is provided below:

(SEQ ID NO: 10)
MKPSLLCRPLSCFLMLLPWPLATLTSTTLWQCPPGEEPDLDPGQGTLC
RPCPPGTFSAAWGSSPCQPHARCSLWRRLEAQVGMATRDTLCGDCWPG
WFGPWGVPRVPCQPCSWAPLGTHGCDEWGRRARRGVEVAAGASSGGET
RQPGNGTRAGGPEETAAQYAVIAIVPVFCLMGLLGILVCNLLKRKGYH
CTAHKEVGPGPGGGGSGINPAYRTEDANEDTIGVLVRLITEKKENAAA
LEELLKEYHSKQLVQTSHRPVSKLPPAPPNVPHICPHRHHLHTVQGLA
SLSGPCCSRCSQKKWPEVLLSPEAVAATTPVPSLLPNPTRVPKAGAKA
GRQGEITILSVGRFRVARIPEQRTSSMVSEVKTITEAGPSWGDLPDSP
QPGLPPEQQALLGSGGSRTKWLKPPAENKAEENRYVVRLSESNLVI

TNF-RSF19L sequences can also be found in publicly available databases, for example, GenBank, OMIM, and UniProt (Q969Z4).

### TNF-RSF27

Human TNF-RSF27 protein, also known as ectodysplasin A2 receptor, EDA2R, XEDAR, EDAA2R, EDA-A2R and TNFRSF27 is a type III transmembrane protein of the TNFR (tumor necrosis factor receptor) superfamily, and contains cysteine-rich repeats and a single transmembrane domain. It binds to the EDA-A2 isoform of ectodysplasin, which plays an important role in maintenance of hair and teeth. Loss- and gain-of-function studies have indicated that EDA, particularly the EDA-A2 isoform, regulates systemic glucose metabolism in type 2 diabetes mellitus (T2DM) (Awazawa et al., Nat. Med., 2017, 23, 1466-1473).

The amino acid sequence of human TNF-RSF27 (NP_001186616.2) is provided below:

(SEQ ID NO: 11)
MDCQENEYWDQWGRCVTCQRCGPGQELSKDCGYGEGGDAYCTACPPRR
YKSSWGHHRCQSCITCAVINRVQKVNCTATSNAVCGDCLPRFYRKTRI
GGLQDQECIPCTKQTPTSEVQCAFQLSLVEADTPTVPPQEATLVALVS
SLLVVFTLAFLGLFFLYCKQFFNRHCQRGGLLQFEADKTAKEESLFPV
PPSKETSAESQVSENIFQTQPLNPILEDDCSSTSGFPTQESFTMASCT
SESHSHWVHSPIECTELDLQKFSSSASYTGAETLGGNTVESTGDRLEL
NVPFEVPSP

TNF-RSF27 sequences can also be found in publicly available databases, for example, GenBank, OMIM, and UniProt (Q9HAV5).

### IL-1RT1

Human IL-1RT1, also known as interleukin 1 receptor type 1 (also refereed to as IL1R1, P80, IL1R, IL1RA, CD121A, D2S1473, and IL-1R-alpha), is a cytokine receptor that belongs to the interleukin-1 receptor family. It is a receptor for IL-1α and IL-1β, and interleukin-1 receptor antagonist. IL-1RT1 is an important mediator involved in many cytokine-induced immune and inflammatory responses. The binding of IL-1α or IL-1β triggers the dimerization with IL-1R receptor accessory protein (IL-1RAP), resulting in activation of NF-κB (Buzzelli et al., Oncotarget, 2015, 6(2): 679-695).

The amino acid sequence of human IL-1RT1 (NP_000868.1) is provided below:

(SEQ ID NO: 12)
MKVLLRLICFIALLISSLEADKCKEREEKIILVSSANEIDVRPCPLNP
NEHKGTITWYKDDSKTPVSTEQASRIHQHKEKLWFVPAKVEDSGHYYC
VVRNSSYCLRIKISAKFVENEPNLCYNAQAIFKQKLPVAGDGGLVCPY
MEFFKNENNELPKLQWYKDCKPLLLDNIHFSGVKDRLIVMNVAEKHRG
NYTCHASYTYLGKQYPITRVIEFITLEENKPTRPVIVSPANETMEVDL
GSQIQLICNVTGQLSDIAYWKWNGSVIDEDDPVLGEDYYSVENPANKR
RSTLITVLNISEIESRFYKHPFTCFAKNTHGIDAAYIQLIYPVTNFQK
HMIGICVTLTVIIVCSVFIYKIFKIDIVLWYRDSCYDFLPIKASDGKT
YDAYILYPKTVGEGSTSDCDIFVFKVLPEVLEKQCGYKLFIYGRDDYV
GEDIVEVINENVKKSRRLIIILVRETSGFSWLGGSSEEQIAMYNALVQ
DGIKVVLLELEKIQDYEKMPESIKFIKQKHGAIRWSGDFTQGPQSAKT
RFWKNVRYHMPVQRRSPSSKHQLLSPATKEKLQREAHVPLG

IL-1RT1 sequences can also be found in publicly available databases, for example, GenBank, OMIM, and UniProt (P14778).

### CD160

Human CD160, also known as CD160 molecule, NK1, BY55 and NK28, is a cell-surface glycoprotein expressed on natural killer (NK) cells, CD8+ cells, a small subset of CD4+ cells, and all intraepithelial lymphocytes (IELs). Its expression is tightly associated with peripheral blood NK cells and CD8 T lymphocytes with cytolytic effector activity. A soluble form of CD160, shed from NK cells, has also been reported to inhibit cell-mediated cytotoxicity (Liu et al., Structure, 2019, 27(8), 1286-1295.e4). Human CD160 also binds to herpesvirus entry mediator (HVEM), a TNF family member, with much higher affinity than to MHC class I, and leads to suppressed T cell responses in vitro (Tu et al., J Exp Med, 2015, 212 (3): 415-429).

The amino acid sequence of human CD160 (NP_008984.1) is provided below:

(SEQ ID NO: 13)
MLLEPGRGCCALAILLAIVDIQSGGCINITSSASQEGTRLNLICTVWH
KKEEAEGFVVFLCKDRSGDCSPETSLKQLRLKRDPGIDGVGEISSQLM
FTISQVTPLHSGTYQCCARSQKSGIRLQGHFFSILFTETGNYTVTGLK
QRQHLEFSHNEGTLSSGFLQEKVWVMLVTSLVALQAL

CD160 sequences can also be found in publicly available databases, for example, GenBank, OMIM, and UniProt (095971).

### EPHA2

Human EPHA2, also known as EPH receptor A2, ECK, CTPA, ARCC2, CTPP1 and CTRCT6, is a member of the largest family of receptor tyrosine kinases (RTKs)—the Eph receptor subfamily of the protein-tyrosine kinase family. EphA2 plays a role in lens, kidney, bone, mammary gland and ear development (Park et al., Genes, 2013, 4, 334-357, doi:10.3390/genes4030334).

The amino acid sequence of human EPHA2 (NP_001316019.1) is provided below:

(SEQ ID NO: 14)
MQNIMNDMPIYMYSVCNVMSGDQDNWLRTNWVYRGEAERIFIELKFTVRDCNSFPGG
ASSCKETFNLYYAESDLDYGTNFQKRLFTKIDTIAPDEITVSSDFEARHVKLNVEERSVG
PLTRKGFYLAFQDIGACVALLSVRVYYKKCPELLQGLAHFPETIAGSDAPSLATVAGTC
VDHAVVPPGGEEPRMHCAVDGEWLVPIGQCLCQAGYEKVEDACQACSPGFFKFEASES
PCLECPEHTLPSPEGATSCECEEGFFRAPQDPASMPCTRPPSAPHYLTAVGMGAKVELR
WTPPQDSGGREDIVYSVTCEQCWPESGECGPCEASVRYSEPPHGLTRTSVTVSDLEPHM
NYTFTVEARNGVSGLVTSRSFRTASVSINQTEPPKVRLEGRSTTSLSVSWSIPPPQQSRVW
KYEVTYRKKGDSNSYNVRRTEGFSVTLDDLAPDTTYLVQVQALTQEGQGAGSKVHEFQ
TLSPEGSGNLAVIGGVAVGVVLLLVLAGVGFFIHRRRKNQRARQSPEDVYFSKSEQLKP
LKTYVDPHTYEDPNQAVLKFTTEIHPSCVTRQKVIGAGEFGEVYKGMLKTSSGKKEVPV
AIKTLKAGYTEKQRVDFLGEAGIMGQFSHHNIIRLEGVISKYKPMMIITEYMENGALDKF
LREKDGEFSVLQLVGMLRGIAAGMKYLANMNYVHRDLAARNILVNSNLVCKVSDFGL
SRVLEDDPEATYTTSGGKIPIRWTAPEAISYRKFTSASDVWSFGIVMWEVMTYGERPYW
ELSNHEVMKAINDGFRLPTPMDCPSAIYQLMMQCWQQERARRPKFADIVSILDKLIRAP
DSLKTLADFDPRVSIRLPSTSGSEGVPFRTVSEWLESIKMQQYTEHFMAAGYTAIEKVVQ
MTNDDIKRIGVRLPGHQKRIAYSLLGLKDQVNTVGIPI

EPHA2 sequences can also be found in publicly available databases, for example, GenBank, OMIM, and UniProt (P29317).

### EFNA4

Human EFNA4, also known as ephrin A4, EFL4, EPLG4, LERK4 and LERK-4, is the ligand of EPH family. The ephrins and EPH-related receptors comprise the largest subfamily of receptor protein-tyrosine kinases and have been implicated in mediating developmental events, especially in the nervous system and in erythropoiesis. EFNA4 mainly expresses in the spleen, lymph nodes, ovary, small intestine, and colon of adults, as well as in the heart, lungs, liver, and kidneys of the fetus.

The amino acid sequence of human EFNA4 (NP_005218.1) is provided below:

(SEQ ID NO: 15)
MRLLPLLRTVLWAAFLGSPLRGGSSLRHVVYWNSSNPRLLRGDAVVEL
GLNDYLDIVCPHYEGPGPPEGPETFALYMVDWPGYESCQAEGPRAYKR
WVCSLPFGHVQFSEKIQRFTPFSLGFEFLPGETYYYISVPTPESSGQC
LRLQVSVCCKERKSESAHPVGSPGESGTSGWRGGDTPSPLCLLLLLLL
LILRLLRIL

EFNA4 sequences can also be found in publicly available databases, for example, GenBank, OMIM, and UniProt (P52798).

### GFR-Alpha-1

Human GFR-alpha-1, also known as GDNF family receptor alpha 1, GFRA1, GDNFR, RET1L, RETL1, TRNR1, GDNFRA, and GDNFR-alpha-1, is a glycosylphosphatidylinositol (GPI)-linked cell surface receptor for both glial cell line-derived neurotrophic factor (GDNF) and neurturin (NTN), and mediates activation of the RET tyrosine kinase receptor. GFR-alpha-1 is potentially associated with for Hirschsprung disease (Konishi et al., J Neurosci., 2014, 34(39): 13127-13138).

The amino acid sequence of human GFR-alpha-1 NP_001138925.1 is provided below:

(SEQ ID NO: 16)
MFLATLYFALPLLDLLLSAEVSGGDRLDCVKASDQCLKEQSCSTKYRT
LRQCVAGKETNFSLASGLEAKDECRSAMEALKQKSLYNCRCKRGMKKE
KNCLRIYWSMYQSLQGNDLLEDSPYEPVNSRLSDIFRVVPFISVEHIP
KGNNCLDAAKACNLDDICKKYRSAYITPCTTSVSNDVCNRRKCHKALR
QFFDKVPAKHSYGMLFCSCRDIACTERRRQTIVPVCSYEEREKPNCLN
LQDSCKTNYICRSRLADFFTNCQPESRSVSSCLKENYADCLLAYSGLI
GTVMTPNYIDSSSLSVAPWCDCSNSGNDLEECLKFLNFFKDNTCLKNA
IQAFGNGSDVTVWQPAFPVQTTTATTTTALRVKNKPLGPAGSENEIPT
HVLPPCANLQAQKLKSNVSGNTHLCISNGNYEKEGLGASSHITTKSMA
APPSCGLSPLLVLVVTALSTLLSLTETS

GFR-alpha-1 sequences can also be found in publicly available databases, for example, GenBank, OMIM, and UniProt (P56159).

### WFDC2

Human WFDC2, also known as WAP four-disulfide core domain 2, HE4, WAP5, EDDM4 and dJ461P17.6, is a member of the WFDC domain family. The WFDC domain, or WAP Signature motif, contains eight cysteines forming four disulfide bonds at the core of the protein, and functions as a protease inhibitor in many family members. WFDC2 has been identified as a tumor suppressor which inhibits the metastasis of prostate cancer in vitro and in vivo, and that WFDC2 binds to the extracellular domain of epidermal growth factor receptor (EGFR) (Xiong et al., Cell Death & Disease, 2020, 11, 537).

The amino acid sequence of human WFDC2 (NP_006094.3) is provided below:

(SEQ ID NO: 17)
MPACRLGPLAAALLLSLLLFGFTLVSGTGAEKTGVCPELQADQNCTQE
CVSDSECADNLKCCSAGCATFCSLPNDKEGSCPQVNINFPQLGLCRDQ
CQVDSQCPGQMKCCRNGCGKVSCVTPNF

WFDC2 sequences can also be found in publicly available databases, for example, GenBank, OMIM, and UniProt (Q14508).

### DLL1

Human DLL1, also known as delta like canonical Notch ligand 1, DL1, Delta, DELTA1 and NEDBAS, belongs to the Delta/Jagged family of transmembrane proteins. DLL1 is implicated in regulating cell fate determination, proliferation, stem cell self-renewal and apoptosis. DLL1 may also play a role in cell-to-cell communication (Hildebrand et al., Front Cell Infect Microbiol., 20199: 267).

The amino acid sequence of human DLL1 (NP 005609.3) is provided below:

(SEQ ID NO: 18)
MGSRCALALAVLSALLCQVWSSGVFELKLQEFVNKKGLLGNRNCCRGG
AGPPPCACRTFFRVCLKHYQASVSPEPPCTYGSAVTPVLGVDSFSLPD
GGGADSAFSNPIRFPFGFTWPGTFSLIIEALHTDSPDDLATENPERLI
SRLATQRHLTVGEEWSQDLHSSGRTDLKYSYRFVCDEHYYGEGCSVFC
RPRDDAFGHFTCGERGEKVCNPGWKGPYCTEPICLPGCDEQHGFCDKP
GECKCRVGWQGRYCDECIRYPGCLHGTCQQPWQCNCQEGWGGLFCNQD
LNYCTHHKPCKNGATCTNTGQGSYTCSCRPGYTGATCELGIDECDPSP
CKNGGSCTDLENSYSCTCPPGFYGKICELSAMTCADGPCFNGGRCSDS
PDGGYSCRCPVGYSGENCEKKIDYCSSSPCSNGAKCVDLGDAYLCRCQ
AGFSGRHCDDNVDDCASSPCANGGTCRDGVNDFSCTCPPGYTGRNCSA
PVSRCEHAPCHNGATCHERGHRYVCECARGYGGPNCQFLLPELPPGPA
VVDLTEKLEGQGGPFPWVAVCAGVILVLMLLLGCAAVVVCVRLRLQKH
RPPADPCRGETETMNNLANCQREKDISVSIIGATQIKNTNKKADFHGD
HSADKNGFKARYPAVDYNLVQDLKGDDTAVRDAHSKRDTKCQPQGSSG
EEKGTPTTLRGGEASERKRPDSGCSTSKDTKYQSVYVISEEKDECVIA
TEV

DLL1 sequences can also be found in publicly available databases, for example, GenBank, OMIM, and UniProt (000548).

### LAYN

Human LAYN, also known as layilin, is a C-type lectin domain-containing membrane glycoprotein having about 55 kDa. LAYN is selectively expressed on highly activated, clonally expanded, but phenotypically exhausted CD8+ T cells in human melanoma. Lineage-specific deletion of layilin on murine CD8+ T cells reduced their accumulation in tumors and increased tumor growth in vivo (Mahuron et al., J Exp Med, 2020, 217 (9): e20192080).

The amino acid sequence of human LAYN (NP_001245319.1) is provided below:

(SEQ ID NO: 19)
MRPGTALQAVLLAVLLVGLRAATGRLLSASDLDLRGGQPVCRGGTQRP
CYKVIYFHDTSRRLNFEEAKEACRRDGGQLVSIESEDEQKLIEKFIEN
LLPSDGDFWIGLRRREEKQSNSTACQDLYAWTDGSISQFRNWYVDEPS
CGSEVCVVMYHQPSAPAGIGGPYMFQWNDDRCNMKNNFICKYSDEKPA
VPSREAEGEETELTTPVLPEETQEEDAKKTFKESREAALNLAYILIPS
IPLLLLLVVTTVVCWVWICRKRKREQPDPSTKKQHTIWPSPHQGNSPD
LEVYNVIRKQSEADLAETRPDLKNISFRVCSGEATPDDMSCDYDNMAV
NPSESGFVTLVSVESGFVTNDIYEFSPDQMGRSKESGWVENEIYGY

LAYN sequences can also be found in publicly available databases, for example, GenBank, OMIM, and UniProt (Q6UX15).

### PVRL4

Human PVRL4, also known as NECTIN4, nectin cell adhesion molecule 4, LNIR, PRR4, EDSS1 and nectin-4, is a member of the nectin family. The PVRL4 contains two immunoglobulin-like (Ig-like) C2-type domains and one Ig-like V-type domain. It is involved in cell adhesion through trans-homophilic and -heterophilic interactions. PVRL4 is a single-pass type I membrane protein.

The amino acid sequence of human PVRL4 (NP_112178.2) is provided below:

(SEQ ID NO: 20)
MPLSLGAEMWGPEAWLLLLLLLASFTGRCPAGELETSDVVTVVLGQDA
KLPCFYRGDSGEQVGQVAWARVDAGEGAQELALLHSKYGLHVSPAYEG
RVEQPPPPRNPLDGSVLLRNAVQADEGEYECRVSTFPAGSFQARLRLR
VLVPPLPSLNPGPALEEGQGLTLAASCTAEGSPAPSVTWDTEVKGTTS
SRSFKHSRSAAVTSEFHLVPSRSMNGQPLTCVVSHPGLLQDQRITHIL
HVSFLAEASVRGLEDQNLWHIGREGAMLKCLSEGQPPPSYNWTRLDGP
LPSGVRVDGDTLGFPPLTTEHSGIYVCHVSNEFSSRDSQVTVDVLDPQ
EDSGKQVDLVSASVVVVGVIAALLFCLLVVVVVLMSRYHRRKAQQMTQ
KYEEELTLTRENSIRRLHSHHTDPRSQPEESVGLRAEGHPDSLKDNSS
CSVMSEEPEGRSYSTLTTVREIETQTELLSPGSGRAEEEEDQDEGIKQ
AMNHFVQENGTLRAKPTGNGIYINGRGHLV

PVRL4 sequences can also be found in publicly available databases, for example, GenBank, OMIM, and UniProt (Q96NY8).

### PI3

Human PI3, also known as Peptidase Inhibitor 3, ESI, WAP3, SKALP, WFDC14 and cementoin, is an epithelial protein that is secreted by keratinocytes in response to IL-1 and TNFα. It contains a WAP-type four-disulfide core (WFDC) domain, and is thus a member of the WFDC domain family. PI3 is also shown to be protective against several lung and cardiovascular injuries, including hypoxia-induced pulmonary hypertension, viral myocarditis, and vascular injury in elafin-overexpressing transgenic mice (Li et al., Journal of Interferon & Cytokine Research, 2020, 40:6).

The amino acid sequence of human PI3 (NP_002629.1) is provided below:

(SEQ ID NO: 21)
MRASSFLIVVVFLIAGTLVLEAAVTGVPVKGQDTVKGRVPFNGQDPVK
GQVSVKGQDKVKAQEPVKGPVSTKPGSCPIILIRCAMLNPPNRCLKDT
DCPGIKKCCEGSCGMACFVPQ

PI3 sequences can also be found in publicly available databases, for example, GenBank, OMIM, and UniProt (P19957).

### SYND1

Human SYND1, also known as SDC1, syndecan 1, SDC, CD138 and syndecan, is a transmembrane (type I) heparan sulfate proteoglycan and is a member of the syndecan proteoglycan family. The syndecans mediate cell binding, cell signaling, and cytoskeletal organization and syndecan receptors are required for internalization of the HIV-1 tat protein. SYND1 functions as an integral membrane protein and participates in cell proliferation, cell migration and cell-matrix interactions via its receptor for extracellular matrix proteins. Altered SYND1 expression has been detected in several different tumor types (Szatmári et al., Dis Markers, 2015, v2015: 796052). For example, SYND1 is a critical mediator of macropinocytosis in pancreatic cancer (Yao et al., Nature, 2019, 568: 410-414).

The amino acid sequence of human SYND1 (NP_001006947.2) is provided below:

(SEQ ID NO: 22)
MRRAALWLWLCALALSLQPALPQIVATNLPPEDQDGSGDDSDNFSGSG
AGALQDITLSQQTPSTWKDTQLLTAIPTSPEPTGLEATAASTSTLPAG
EGPKEGEAVVLPEVEPGLTAREQEATPRPRETTQLPTTHLASTTTATT
AQEPATSHPHRDMQPGHHETSTPAGPSQADLHTPHTEDGGPSATERAA
EDGASSQLPAAEGSGEQDFTFETSGENTAVVAVEPDRRNQSPVDQGAT
GASQGLLDRKEVLGGVIAGGLVGLIFAVCLVGFMLYRMKKKDEGSYSL
EEPKQANGGAYQKPTKQEEFYA

SYND1 sequences can also be found in publicly available databases, for example, GenBank, OMIM, and UniProt (P18827).

### KIM1

Human KIM1, also known as TIM, TIM1, CD365, HAVCR, KIM-1, TIM-1, TIMD1, TIMD-1, and HAVCR-1, is a membrane receptor for both human hepatitis A virus (HHAV) and TIMD4. KIM1 may be involved in the moderation of asthma and allergic diseases. KIM1 is upregulated in renal tubular cells after ischemic injury. It is not expressed in the normal kidney but is expressed in a variety of human kidney diseases, predominantly in the apical membrane of proximal tubular cells (Griffin et al, Am J Nephrol. 2020, 51(6): 473-479).

The amino acid sequence of human KIM1 (NP_001166864.1) is provided below:

(SEQ ID NO: 23)
MHPQVVILSLILHLADSVAGSVKVGGEAGPSVTLPCHYSGAVTSMCWN
RGSCSLFTCQNGIVWTNGTHVTYRKDTRYKLLGDLSRRDVSLTIENTA
VSDSGVYCCRVEHRGWFNDMKITVSLEIVPPKVTTTPIVTTVPTVTTV
RTSTTVPTTTTVPMTTVPTTTVPTTMSIPTTTTVLTTMTVSTTTSVPT
TTSIPTTTSVPVTTTVSTFVPPMPLPRQNHEPVATSPSSPQPAETHPT
TLQGAIRREPTSSPLYSYTTDGNDTVTESSDGLWNNNQTQLFLEHSLL
TANTTKGIYAGVCISVLVLLALLGVIIAKKYFFKKEVQQLSVSFSSLQ
IKALQNAVEKEVQAEDNIYIENSLYATD

KIM1 sequences can also be found in publicly available databases, for example, GenBank, OMIM, and UniProt (Q96D42).

### MEP1B

Human MEP1B, also known as meprin A subunit beta, is the beta subunit of a meprin protein. Meprins are multidomain zinc metalloproteases that are highly expressed in mammalian kidney and intestinal brush border membranes, and in leukocytes and certain cancer cells. Targeted disruption of MEP1B in mice affects embryonic viability, renal gene expression profiles, and distribution of the membrane-associated alpha subunit in kidney and intestine (Kaushal et al, Am J Physiol Renal Physiol., 2013, 304(9): F1150-F1158).

The amino acid sequence of human MEP1B (NP_001295100.1) is provided below:

(SEQ ID NO: 24)
MDLWNLSWFLFLDALLVISGLATPENFDVDGGMDQDIFDINEGLGLDL
FEGDIRLDRAQIRNSIIGEKYRWPHTIPYVLEDSLEMNAKGVILNAFE
RYRLKTCIDFKPWAGETNYISVFKGSGCWSSVGNRRVGKQELSIGANC
DRIATVQHEFLHALGFWHEQSRSDRDDYVRIMWDRILSGREHNFNTYS
DDISDSLNVPYDYTSVMHYSKTAFQNGTEPTIVTRISDFEDVIGQRMD
FSDSDLLKLNQLYNCSSSLSFMDSCSFELENVCGMIQSSGDNADWQRV
SQVPRGPESDHSNMGQCQGSGFFMHFDSSSVNVGATAVLESRTLYPKR
GFQCLQFYLYNSGSESDQLNIYIREYSADNVDGNLTLVEEIKEIPTGS
WQLYHVTLKVTKKFRVVFEGRKGSGASLGGLSIDDINLSETRCPHHIW
HIRNFTQFIGSPNGTLYSPPFYSSKGYAFQIYLNLAHVTNAGIYFHLI
SGANDDQLQWPCPWQQATMTLLDQNPDIRQRMSNQRSITTDPFMTTDN
GNYFWDRPSKVGTVALFSNGTQFRRGGGYGTSAFITHERLKSRDFIKG
DDVYILLTVEDISHLNSTQIQLTPAPSVQDLCSKTTCKNDGVCTVRDG
KAECRCQSGEDWWYMGERCEKRGSTRDTIVIAVSSTVAVFALMLIITL
VSVYCTRKKYRERMSSNRPNLTPQNHAF

MEP1B sequences can also be found in publicly available databases, for example, GenBank, OMIM, and UniProt (Q16820).

### PILRB

Human PILRB is also known as paired immunoglobin like type 2 receptor beta, FDFACT1 and FDFACT2. The paired immunoglobin-like type 2 receptors consist of highly related activating and inhibitory receptors that are involved in the regulation of many aspects of the immune system. PILRB is the activating member of the receptor pair and contains a truncated cytoplasmic tail relative to its inhibitory counterpart (PILRA), that has a long cytoplasmic tail with immunoreceptor tyrosine-based inhibitory (ITIM) motifs.

The amino acid sequence of human PILRB (NP_001358860.1) is provided below:

(SEQ ID NO: 25)
MGRPLLLPLLLLLQPPAFLQPGGSTGSGPSYLYGVTQPKHLSASMGGS
VEIPFSFYYPWELAIVPNVRISWRRGHFHGQSFYSTRPPSIHKDYVNR
LFLNWTEGQESGFLRISNLRKEDQSVYFCRVELDTRRSGRQQLQSIKG
TKLTITQAVTTTTTWRPSSTTTIAGLRVTESKGHSESWHLSLDTAIRV
ALAVAVLKTVILGLLCLLLLWWRRRKGLSHRTRPVWNLLYGRSMCGIS
AELSIGNLDVHISSSKERTRLQAAMSSTSISSKPDSSRAPSSDF

PILRB sequences can also be found in publicly available databases, for example, GenBank, OMIM, and UniProt (Q9UKJ0).

### GDF15

Human GDF15, also known as growth differentiation factor 15, PDF, MIC1, PLAB, MIC-1, NAG-1, PTGFB, and GDF-15, is a secreted ligand of the TGFβ (transforming growth factor-beta) superfamily of proteins. Ligands of this family bind various TGFβ receptors leading to recruitment and activation of SMAD family transcription factors that regulate gene expression. Functional studies demonstrate that GDF15 deficiency enhanced renal tubular injury and inflammation post-IRI. Further, genetic association studies suggest that lower circulating GDF15 levels link to an elevated risk of rejection in patients receiving kidney transplant (Liu et al., JASN, 2020, 31 (4): 701-715; DOI: https://doi.org/10.1681/ASN.2019090876).

The amino acid sequence of human GDF15 (NP_004855.2) is provided below:

(SEQ ID NO: 26)
MPGQELRTVNGSQMLLVLLVLSWLPHGGALSLAEASRASFPGPSELHS
EDSRFRELRKRYEDLLTRLRANQSWEDSNTDLVPAPAVRILTPEVRLG
SGGHLHLRISRAALPEGLPEASRLHRALFRLSPTASRSWDVTRPLRRQ
LSLARPQAPALHLRLSPPPSQSDQLLAESSSARPQLELHLRPQAARGR
RRARARNGDHCPLGPGRCCRLHTVRASLEDLGWADWVLSPREVQVTMC
IGACPSQFRAANMHAQIKTSLHRLKPDTVPAPCCVPASYNPMVLIQKT
DTGVSLQTYDDLLAKDCHCI

GDF15 sequences can also be found in publicly available databases, for example, GenBank, OMIM, and UniProt (Q99988).

### ANGPT1

ANGPT1, also known as Angiopoietin 1, KIAA0003, ANG-1, AGP1, and AGPT, is a secreted 70-kDa glycoprotein and a member of the angiopoietin family of growth factors. ANGPT1 is the major agonist for the tyrosine kinase receptor, Tek, which is found primarily on endothelial cells. ANGPT1 is produced by vasculature support cells and specialized pericytes such as podocytes in the kidney and ITO cells in the liver (Satchell et al. J Am Soc Nephrol, 2002, 13(2):544-550). ANGPT1 plays an important role in the regulation of angiogenesis, endothelial cell survival, proliferation, migration, adhesion and cell spreading, reorganization of the actin cytoskeleton, and maintenance of vascular quiescence (Jeansson et al. J Clin Invest, 2011, 121(6): 2278-2289).

The amino acid sequence of human ANGPT1 (NP_001137.2) is provided below:

(SEQ ID NO: 27)
MTVFLSFAFLAAILTHIGCSNQRRSPENSGRRYNRIQHGQCAYTFILP
EHDGNCRESTTDQYNTNALQRDAPHVEPDFSSQKLQHLEHVMENYTQW
LQKLENYIVENMKSEMAQIQQNAVQNHTATMLEIGTSLLSQTAEQTRK
LTDVETQVLNQTSRLEIQLLENSLSTYKLEKQLLQQTNEILKIHEKNS
LLEHKILEMEGKHKEELDTLKEEKENLQGLVTRQTYIIQELEKQLNRA
TTNNSVLQKQQLELMDTVHNLVNLCTKEGVLLKGGKREEEKPFRDCAD
VYQAGFNKSGIYTIYINNMPEPKKVFCNMDVNGGGWTVIQHREDGSLD
FQRGWKEYKMGFGNPSGEYWLGNEFIFAITSQRQYMLRIELMDWEGNR
AYSQYDRFHIGNEKQNYRLYLKGHTGTAGKQSSLILHGADFSTKDADN
DNCMCKCALMLTGGWWFDACGPSNLNGMFYTAGQNHGKLNGIKWHYFK
GPSYSLRSTTMMIRPLDF

Further examples of ANGPT1 sequences can be found in publicly available databases, for example, GenBank, OMIM, and UniProt (Q15389).

### ANGPT2

Human ANGPT2, also known as angiopoietin 2, ANG2, AGPT2 and LMPHM10, belongs to the angiopoietin family of growth factors. ANGPT2 is an antagonist of angiopoietin 1, and both angiopoietin 1 and angiopoietin 2 are ligands for the endothelial TEK receptor tyrosine kinase. ANGPT2 is associated with kidney disease. For example, ANGPT2 can induce arterial stiffness in chronic kidney disease (Chang et al., J Am Soc Nephrol., 2014, 25(6): 1198-1209). Furthermore, high ANGPT2 and low ANGPT1 are positively associated with abnormal cardiac structure in stages 3-5 chronic kidney disease patients (Tsai et al., Scientific Reports, 2016, 6:39400).

The amino acid sequence of human ANGPT2 (NP_001112359.1) is provided below:

(SEQ ID NO: 28)
MWQIVFFTLSCDLVLAAAYNNFRKSMDSIGKKQYQVQHGSCSYTFLLP
EMDNCRSSSSPYVSNAVQRDAPLEYDDSVQRLQVLENIMENNTQWLMK
LENYIQDNMKKEMVEIQQNAVQNQTAVMIEIGTNLLNQTAEQTRKLTD
VEAQVLNQTTRLELQLLEHSLSTNKLEKQILDQTSEINKLQDKNSFLE
KKVLAMEDKHIIQLQSIKEEKDQLQVLVSKQNSIIEELEKKIVTATVN
NSVLQKQQHDLMETVNNLLTMMSTSNSKDPTVAKEEQISFRDCAEVFK
SGHTTNGIYTLTFPNSTEEIKAYCDMEAGGGGWTIIQRREDGSVDFQR
TWKEYKVGFGNPSGEYWLGNEFVSQLTNQQRYVLKIHLKDWEGNEAYS
LYEHFYLSSEELNYRIHLKGLTGTAGKISSISQPGNDFSTKDGDNDKC
ICKCSQMLTGGWWFDACGPSNLNGMYYPQRQNTNKFNGIKWYYWKGSG
YSLKATTMMIRPADF

ANGPT2 sequences can also be found in publicly available databases, for example, GenBank, OMIM, and UniProt (015123).

### TNFSF12

Human TNFSF12, also known as Tumor Necrosis Factor Superfamily Member 12, APO3L, DR3LG, TWEAK, and TNLG4A, is a member of the tumor necrosis factor (TNF) family of proteins that play pivotal roles in the regulation of the immune system. TNFSF12 is expressed widely in many tissues and induces interleukin-8 synthesis in a number of cell lines (Chicheportiche et al. Cell Biology and Metabolism, 1997, 272(51): 32401-32410). TNFSF12 suppresses production of IFN-γ and IL-12, curtailing the innate response and its transition to adaptive TH1 immunity. TNFSF12 also promotes proliferation and migration of endothelial cells, acting as a regulator of angiogenesis.

The amino acid sequence of human TNFSF12 (NP_003800.1) is provided below:

(SEQ ID NO: 29)
MAARRSQRRRGRRGEPGTALLVPLALGLGLALACLGLLLAVVSLGSRA
SLSAQEPAQEELVAEEDQDPSELNPQTEESQDPAPFLNRLVRPRRSAP
KGRKTRARRAIAAHYEVHPRPGQDGAQAGVDGTVSGWEEARINSSSPL
RYNRQIGEFIVTRAGLYYLYCQVHFDEGKAVYLKLDLLVDGVLALRCL
EEFSATAASSLGPQLRLCQVSGLLALRPGSSLRIRTLPWAHLKAAPFL
TYFGLFQVH

Further examples of TNFSF12 sequences can be found in publicly available databases, for example, GenBank, OMIM, and UniProt (043508).

### LRP11

Human LRP11, also known as LDL receptor related protein 11, MANSC3 and bA350J20.3, is predicted to act upstream of or within several processes, including response to cold; response to immobilization stress; and response to water deprivation. LRP11 may play an important role in proliferation, migration and invasion of cervical cancer (Wang et al., Cancer Manag Res., 2019, 11: 8081-8093).

The amino acid sequence of human LRP11 (NP_116221.3) is provided below:

(SEQ ID NO: 30)
MASVAQESAGSQRRLPPRHGALRGLLLLCLWLPSGRAALPPAAPLSEL
HAQLSGVEQLLEEFRRQLQQERPQEELELELRAGGGPQEDCPGPGSGG
YSAMPDAIIRTKDSLAAGASFLRAPAAVRGWRQCVAACCSEPRCSVAV
VELPRRPAPPAAVLGCYLFNCTARGRNVCKFALHSGYSSYSLSRAPDG
AALATARASPRQEKDAPPLSKAGQDVVLHLPTDGVVLDGRESTDDHAI
VQYEWALLQGDPSVDMKVPQSGTLKLSHLQEGTYTFQLTVTDTAGQRS
SDNVSVTVLRAAYSTGGCLHTCSRYHFFCDDGCCIDITLACDGVQQCP
DGSDEDFCQNLGLDRKMVTHTAASPALPRTTGPSEDAGGDSLVEKSQK
ATAPNKPPALSNTEKRNHSAFWGPESQIIPVMPDSSSSGKNRKEESYI
FESKGDGGGGEHPAPETGAVLPLALGLAITALLLLMVACRLRLVKQKL
KKARPITSEESDYLINGMYL

LRP11 sequences can also be found in publicly available databases, for example, GenBank, OMIM, and UniProt (Q86VZ4).

### Testican

Human Testican includes Testican-1 and Testican-2. Human Testican-2 protein is encoded by the SPOCK2 gene, also known as TICN2 or KIAA0275. Testican-2 binds with glycosaminoglycans to form part of the extracellular matrix. The protein contains thyroglobulin type-1, follistatin-like, and calcium-binding domains, and has glycosaminoglycan attachment sites in the acidic C-terminal region. SPOCK (SPARC/osteonectin CWCV and Kazal-like domains) encodes a secreted proteoglycan with three known homologs, SPOCK1, -2, and -3. SPOCK was initially characterized as a progenitor form of a seminal plasma GAG-bearing peptide and was later cloned and identified as a chondroitin/heparan sulfate proteoglycan (HSPG).

The amino acid sequence of human Testican-2 (NP_001231879.1, isoform 2 precursor) is provided below:

(SEQ ID NO: 31)
MRAPGCGRLVLPLLLLAAAALAEGDAKGLKEGETPGNFMEDEQWLSSI
SQYSGKIKHWNRFRDEVEDDYIKSWEDNQQGDEALDTTKDPCQKVKCS
RHKVCIAQGYQRAMCISRKKLEHRIKQPTVKLHGNKDSICKPCHMAQL
ASVCGSDGHTYSSVCKLEQQACLSSKQLAVRCEGPCPCPTEQAATSTA
DGKPETCTGQDLADLGDRLRDWFQLLHENSKQNGSASSVAGPASGLDK
SLGASCKDSIGWMFSKLDTSADLFLDQTELAAINLDKYEVCIRPFFNS
CDTYKDGRVSTAEWCFCFWREKPPCLAELERIQIQEAAKKKPGIFIPS
CDEDGYYRKMQCDQSSGDCWCVDQLGLELTGTRTHGSPDCDDIVGFSG
DFGSGVGWEDEEEKETEEAGEEAEEEEGEAGEADDGGYIW

Testican-2 sequences can also be found in publicly available databases, for example, GenBank, OMIM, and UniProt (Q92563).

### NVL1

Human NVL is a member of the AAA (ATPases associated with diverse cellular activities) superfamily, which is characterized by the presence of one (Type I) or two (Type II) conserved ATP-binding modules (AAA modules), each consisting of about 200 amino acid residues. Two alternatively spliced isoforms of NVL, NVL1 (a short isoform) and NVL2 (a long and the main species), are produced through the utilization of different methionines as translation initiation sites. NVL1 is localized to the nucleoplasmin (Nagahama et al., Mol Biol Cell, 2004, 15(12): 5712-5723).

The amino acid sequence of human NVL1 (NP_996671.1, isoform 2) is provided below:

(SEQ ID NO: 32)
MEDYPDPQSANHMNSSLLSLYRKGNPDSVSNTPEMEQRETTSSTPRIS
SKTGSIPLKTPAKDSEGGWFIDKTPSVKKDSFFLDLSCEKSNPKKPIT
EIQDSKDSSLLESDMKRKGKLKNKGSKRKKEDLQEVDGEIEAVLQKKA
KARGLEFQISNVKFEDVGGNDMTLKEVCKMLIHMRHPEVYHHLGVVPP
RGVLLHGPPGCGKTLLAHAIAGELDLPILKVAAPEIVSGVSGESEQKL
RELFEQAVSNAPCIIFIDEIDAITPKREVASKDMERRIVAQLLTCMDD
LNNVAATARVLVIGATNRPDSLDPALRRAGRFDREICLGIPDEASRER
ILQTLCRKLRLPQAFDFCHLAHLTPGFVGADLMALCREAAMCAVNRVL
MKLQEQQKKNPEMEDLPSKGVQEERLGTEPTSETQDELQRLLGLLRDQ
DPLSEEQMQGLCIELNDFIVALSSVQPSAKREGFVTVPNVTWADIGAL
EDIREELTMAILAPVRNPDQFKALGLVTPAGVLLAGPPGCGKTLLAKA
VANESGLNFISVKGPELLNMYVGESERAVRQVFQRAKNSAPCVIFFDE
VDALCPRRSDRETGASVRVVNQLLTEMDGLEARQQVFIMAATNRPDII
DPAILRPGRLDKTLFVGLPPPADRLAILKTITKNGTKPPLDADVNLEA
IAGDLRCDCYTGADLSALVREASICALRQEMARQKSGNEKGELKVSHK
HFEEAFKKVRSSISKKDQIMYERLQESLSR

NVL1 sequences can also be found in publicly available databases, for example, GenBank, OMIM, and UniProt (015381-2).

### Precision Methods

The invention provides methods of assaying a level of a renal-associated protein described herein from a biological sample of a subject, e.g., a plasma sample, and using the level to determine if the subject will be a responder or a non-responder to a given therapy for treating or preventing progressive kidney function decline.

In one embodiment, the invention provides a method for determining whether a human subject will respond to a reno-protective agent for treating or preventing progressive kidney function decline. The method comprises detecting the level of a renal associated protein in a biological sample from the human subject. Renal associated proteins that can be used alone or in combination to determine if the subject is a responder (or a non-responder) include TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof. The method further comprises comparing the level of the renal associated protein with a responder control level. The human subject is a responder to the reno-protective agent if the level of the renal associated protein is equal to or higher than a responder control level, whereas the human subject is not a responder to the reno-protective agent if the level of the renal associated protein is less than the responder control level.

In certain embodiments, the responder control level is a pre-determined and is the average of the level of the renal associated protein in known population of subjects who are responsive to the reno-protective agent.

Generally, the invention includes comparing protein levels from a patient having or at risk of having progressive kidney function decline, with a known standard level associated with disease activity, to determine whether the patient's biomarker level is increased, decreased, or the same, relative to the control. In determining the efficacy of a reno-protective agent for treating kidney disease in a patient, biomarker levels may be pre-determined, or, alternatively, may include obtaining a sample from the patient and then using the biomarker level determined from the sample in the comparative assessment of the invention.

The invention identifies certain biomarkers associated with a therapeutic response, which may be used to determine whether the elected therapeutic agent is adequate for treatment or whether a different therapy, including a different therapeutic agent, should be considered. Such predictive means benefit the overall health of the subject, as faster responses can be made to determine the appropriate therapy. The methods described herein also decrease the overall cost of the treatment process by more quickly eliminating ineffective therapies.

In one aspect, the methods described herein involve detecting the level of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more of the renal associated proteins including, TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican and NVL1.

In some embodiments, the methods comprise comparing the level of the at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more of the renal associated proteins with a responder control level. Accordingly, the subject is determined as a responder to a reno-protective agent if at least one level of the renal associated proteins is equal to or higher than a responder control level, and as not a responder to a reno-protective agent if at least one level of the renal associated proteins is less than a responder control level

In some other embodiments, the methods comprise comparing the level of the at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more of the renal associated proteins with a non-responder control level. Accordingly, the subject is determined as a non-responder to a reno-protective agent if at least one level of the renal associated proteins is equal to or higher than a non-responder control level, and as not a non-responder to a reno-protective agent if at least one level of the renal associated proteins is less than a non-responder control level.

### Methods of Measuring Proteins

The level of a renal associated protein, or combinations thereof, is determined from a biological sample from the subject. Where the level of a renal associated protein is described herein, it is aloes intended that more than one level may be determined for the protein, as well that levels of more than one protein can be measured and used in the analysis to determine if the patient is a non-responder or a responder.

The level of a renal associated protein can be determined using assays known in the art, including, but not limited to, the Slow Off-rate Modified Aptamer (SOMA) scan platform, the OLINK Proximity Extension Assay based proteomic platform, an immunoassay, an ELISA, a western blot, a microarray analysis, a mass spectrometry, a mass spectrometry matrix assisted laser desorption ionization-time-of-flight (MALDI-TOF), an inductively coupled plasma mass spectrometry (ICP-MS), a triggered-by-offset, multiplexed, accurate-mass, high-resolution, and absolute quantification (TOMAHAQ), a direct analysis in real time mass spectrometry (DART-MS), a secondary ion mass spectrometry (SIMS), a liquid chromatography (LC) fractionation, a Mesoscale platform.

In one embodiment, the level of the renal associated protein is determined using immunohistochemical and/or Western analysis, quantitative blood based assays, e.g., serum ELISA, and quantitative urine based assays, e.g., urine ELISA. In one embodiment, an immunoassay is performed to provide a quantitative assessment of the renal associated protein.

The levels of renal associated proteins biomarker may be determined by detecting or quantifying the corresponding expressed polypeptide. The polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, and Western blotting.

Renal associated proteins from biological samples of the subject can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

In one embodiment, the level of renal associated proteins may be determined using an immunoassay. The use of antibodies directed to renal associated protein described herein can be used to screen biological samples, e.g., fluids, for the levels of the renal associated protein, i.e., any one of TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof. By way of illustration, human fluids, such as blood serum or urine, can be taken from a subject and assayed for a specific epitope, either as released antigen or membrane-bound on cells in the sample fluid, using anti-biomarker antibodies in standard RIAs or ELISAs, for example, known in the art. The antibodies used in such methods can be monoclonal antibodies.

In immunoassays, the agent for detecting renal associated protein polypeptide may be an antibody capable of binding to the renal associated protein. Antibodies can be polyclonal, or more preferably, monoclonal. An intact antibody, or a fragment thereof (e.g., Fab or F(ab′)2) can be used.

In one embodiment, antibodies directed to any one of TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or the combination thereof, are used in immunoassays, e.g., ELISA, to determine the level of the same renal associated protein in a sample from a subject. In some embodiments, the level of renal associated protein may be measured in solid tissue (as from a fresh, frozen and/or preserved organ or tissue sample or biopsy or aspirate), whole blood or any blood constituents, or bodily fluids, such as serum, plasma, urine, saliva, sweat or synovial fluid from a subject.

Competitive binding assays may be used to determine the level of the renal associated protein. One example of a competitive binding assay is an enzyme-linked immunosorbent sandwich assay (ELISA). ELISA can be used to detect the presence of renal associated protein in a sample. ELISA is a sensitive immunoassay that uses an enzyme linked to an antibody or antigen as a marker for the detection of a specific protein, especially an antigen or antibody. ELISA is an assay wherein bound antigen or antibody is detected by a linked enzyme that generally converts a colorless substrate into a colored product, or a product which can be detected. One of the most common types of ELISA is “sandwich ELISA”. In one embodiment, the level of the renal associated protein is determined using an ELISA assay.

In addition, a skilled artisan can readily adapt known protein/antibody detection methods for use in determining the amount of a renal associated protein of the present invention.

In one embodiment, antibodies, or antibody fragments, are used in methods such as Western blots or immunofluorescence techniques to detect the renal associated protein. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

Other standard methods using antibodies to detect and quantitate renal associated proteins include, but are not limited to, radioimmunoassays (“RIA”), receptor assays, enzyme immunoassays (“EIA”), cytochemical bioassays, ligand assays, immunoradiometric assays, fluoroimmunoassays, and enzyme-linked immunosorbent assays (“ELISA”). A further method includes, for ease of detection, and its quantitative nature, the sandwich or double antibody assay, of which a number of variations exist, all of which are intended to be encompassed by the present invention. These methods are well known and will be understood by those skilled in the art to require a reasonable amount of experimentation to optimize the interaction between antibodies and antigens and the detection of the antigens by the antibodies. These and other immunoassay techniques may be found in Principles And Practice Of Immunoassay, 2nd Edition, Price and Newman, eds., MacMillan (1997) and Antibodies, A Laboratory Manual, Harlow and Lane, eds., Cold Spring Harbor Laboratory, Ch. 9 (1988), each of which is incorporated herein by reference in its entirety.

Antibodies used in immunoassays known in the art and described herein to determine levels of renal associated protein, may be labeled with a detectable label. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In a one embodiment, the antibody is labeled, e.g. a radio-labeled, chromophore-labeled, fluorophore-labeled, or enzyme-labeled antibody. In another embodiment, an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a renal associated protein.

In one embodiment of the invention, proteomic methods, e.g., mass spectrometry, are used for detecting and quantitating renal associated protein. For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Wright, G. L., Jr., et al. (2002) Expert Rev Mol Diagn 2:549; Li, J., et al. (2002) Clin Chem 48:1296; Laronga, C., et al. (2003) Dis Markers 19:229; Petricoin, E. F., et al. (2002) 359:572; Adam, B. L., et al. (2002) Cancer Res 62:3609; Tolson, J., et al. (2004) Lab Invest 84:845; Xiao, Z., et al. (2001) Cancer Res 61:6029) can be used to detect and quantitate renal associated protein. Mass spectrometric methods are described in, for example, U.S. Pat. Nos. 5,622,824, 5,605,798 and 5,547,835, the entire contents of each of which are incorporated herein by reference.

### Reno-Protective Agents

A reno-protective agent can be used to improve kidney function, e.g., restore kidney function to a healthy kidney level. A reno-protective agent can also be used, in certain embodiments, to maintain kidney function in a human subject in need thereof, for example by slowing kidney function decline such that kidney function no longer declines but is in a steady state. In a further embodiment, a reno-protective agent can be used to minimize decline, e.g., where the rate of decline is reduced. For patients who have progressive kidney decline, a renoprotective agent can be used to slow progress of the patient to end stage kidney disease (ESKD), the final stage of chronic kidney disease

Examples of reno-protective agents for treating or preventing progressive kidney function decline include, but are not limited to, fenofibrate, baricitinib, an SGLT2 inhibitor and a GLP-1/GIP agonist. In some embodiments, the reno-protective agent is fenofibrate. In some other embodiments, the reno-protective agent is baricitinib. In yet some other embodiments, the reno-protective agent is an SGLT2 inhibitor. In yet some other embodiments, the reno-protective agent is a GLP-1/GIP agonist. The reno-protective agent can also be a combination therapy comprising any combination of fenofibrate, baricitinib, SGLT2 inhibitors and GLP-1/GIP agonists. In other embodiments, the reno-protective agent can be a combination of fenofibrate, baricitinib, SGLT2 inhibitors, GLP-1/GIP agonist, the combinations thereof, and with at least one other agent (e.g., metformin).

Fenofibrate is a synthetic phenoxy-isobutyric acid derivate and prodrug with antihyperlipidemic activity. It activates peroxisome proliferator activated receptor α (PPARα). Fenofibrate is mainly used for primary hypercholesterolemia or mixed dyslipidemia. Fenofibrate reduces risk and progression of diabetic retinopathy in type 2 diabetic patients.

Baricitinib, also known as LY3009104, INCB028050 and OLUMIANT® is a Janus kinase (JAK) inhibitor indicated for the treatment of adult patients with moderately to severely active rheumatoid arthritis who have had an inadequate response to one or more TNF antagonist therapies (OLUMIANT FDA label, 2018, pp. 1-19).

Sodium-glucose Cotransporter-2 (SGLT2) inhibitors are a class of prescription medicines that are FDA-approved for use with diet and exercise to lower blood sugar in adults with type 2 diabetes. Medicines in the SGLT2 inhibitor class include, but are not limited to, canagliflozin, dapagliflozin, empagliflozin, ipragliflozin, tofogliflozin, luseogliflozin, remogliflozin etabonate, ertugliflozin, and sotagliflozin. SGLT2 inhibitors are available as single-ingredient products and also in combination with other diabetes medicines such as metformin.

SGLT2 inhibitors have a unique mechanism of action and lower glucose independent of insulin. SGLT1 proteins are expressed in the proximal convoluted tubule of the kidneys. These transporters are an ideal target for the treatment of diabetes because they are responsible for roughly 90% of filtered glucose reabsorption. The normal renal threshold for reabsorption of glucose corresponds to a serum glucose concentration of 180 mg/dL. In patients with type 2 diabetes, this threshold can increase and the expression of the SGLT2 can be up-regulated causing a maladaptive response that worsens hyperglycemia (Moses et al., Australas Med J. 2014, 7(10):405-15). Selective inhibition of SGLT2 inhibitors can reduce this threshold to as low as 40 to 120 mg/dL.

A GLP-1/GIP agonist is a dual agonist for both GLP-1 (glucagon-like peptide-1) and GIP (glucose-dependent insulinotropic polypeptide). GLP-1 stimulates insulin secretion, inhibits glucagon secretion at pancreatic a cells and has also extrapancreatic influences as slowing of gastric emptying which increases the feeling of satiety. GIP is the main incretin hormone in healthy people, causative of most the incretin effects, but the insulin response after GIP secretion in type 2 diabetes mellitus (T2DM) is strongly reduced. An example of a GLP-1/GIP agonist that can be used as a reno-protective agent in accordance with the methods disclosed herein is tirzepatide.

Some further aspects of the present disclosure include treating or preventing a progressive kidney function decline comprising determining whether a subject is a responder to a certain reno-protective agent for treating or preventing progressive kidney function decline, as described herein, and administering a therapeutically effective amount of the reno-protective agent to the responder such that the progressive kidney function decline is treated or prevented. A therapeutically effective amount of the reno-protective agent may be ascertainable by one skilled in the art using known techniques. As is known in the art, adjustments for systemic versus localized delivery, age, body weight, general health, sex, diet, time of administration, drug interaction and the severity of the condition may be necessary, and may be ascertainable with routine experimentation by those skilled in the art. For example, an effective amount of an agent described herein for administration to a subject is an amount that treats or prevents progressive kidney function decline. For example, for any reno-protective agent for treating or preventing progressive kidney function decline, as disclosed herein, a therapeutically effective amount can be an amount that has been shown to provide an observable therapeutic benefit compared to baseline clinically observable signs and symptoms of progressive kidney function decline.

### Kidney Function Decline

There exists a need to establish a meaningful assessment tool for determining if a patient will be responsive to treatment for progressive kidney function decline. Currently, clinicians must provide therapy and wait to determine if it will be effective in the patient. Given the progressive and destructive nature of progressive kidney decline, more efficient means of predicting response to reno-protective agents that can reverse, inhibit, or slow kidney decline are needed.

The invention provides proteins which have been identified as being useful in assessing the ability of a reno-protective agent to treat or prevent progressive kidney function decline. The methods of the invention are advantageous, as they provide a means for the physician to determine the efficacy of a kidney function treatment or prophylactic in a patient without having to wait for clinical outcomes, which may take prolonged periods of time.

Thus, the methods and compositions described herein are useful for treating or preventing progressive kidney function decline. In certain embodiments, the methods disclosed herein can be used to prevent or slow progression of kidney function from normal to chronic kidney disease in a human subject in need thereof. In certain embodiments, the methods disclosed herein can be used to prevent or slow progression of kidney function from chronic kidney disease to end stage kidney disease (ESKD) in a human subject in need thereof.

In one embodiment, the methods and compositions disclosed herein are used to identify a reno-protective agent for a human subject having diabetic kidney disease. Using a renal protective protein level, it can be determined which reno-protective agent will be most effective at treating or preventing progressive kidney function decline in a patient having diabetic kidney disease. Diabetic kidney disease progresses to ESKD, and, therefore, appropriate effective treatment for the subject is key for the subject's overall prognosis.

In some embodiments, the subject is a non-diabetic but has or is at risk of having progressive kidney function decline.

The methods and compositions disclosed herein can be used to predict which reno-protective agent will be best for a patient having kidney disease. Types of kidney disease that can be treated using the methods and compositions disclosed herein are cystinosis, glomerulonephritis, polycystic kidney disease, or IgA nephropathy.

In some embodiments, the present disclosure provides methods of determining whether a subject is predisposed to develop early kidney function decline (EKFD) or has EKFD.

In some embodiments, the subject can have one or more risk factors for developing EKFD, e.g., duration of diabetes, elevated hemoglobin Alec (HbA1c) levels (e.g., above 8.1% or above 9%), age over 35 years, elevated plasma cholesterol levels, high mean blood pressure, elevated albumin to creatinine ratio (e.g., above about 0.6), and hyperglycemia (e.g., blood glucose of over about 200 mg/dL).

In some embodiments, the subject can have microalbuminuria (e.g., excretes 30-300 μg/min albumin). In another aspect, the subject may not have microalbuminuria and/or is a subject with normoalbuminuria (e.g., excretes about less than 30 μg/min) and/or has normal renal function (e.g., has serum creatinine levels at less than 1.2 mg/dl).

In some embodiments, the subject can have type 1 or type 2 diabetes. Alternatively or in addition, the subject can be non-diabetic.

In some embodiments, the subject can have proteinuria, e.g., macroalbuminaria (e.g., the subject excretes more than about 300 micrograms/min albumin).

In some embodiments, the subject does not have, does not have a diagnosis of, or does not present any clinical signs or symptoms of, chronic heart disease (CHD). In some embodiments, the subject does not have, does not have a diagnosis of, or does not present any clinical signs or symptoms of, ischemic heart disease.

In some embodiments, the present disclosure provides methods of determining whether a subject who has or is predisposed to develop end stage kidney disease (ESKD) will be responsive to a therapeutic agent for treatment. In some embodiments, the subject can have one or more risk factors for developing ESKD. Such factors can include, but are not limited to, e.g., duration of diabetes, elevated hemoglobin Ale (HbA1c) levels (e.g., above 8.1% or above 9%), age over 35 years, elevated plasma cholesterol levels, high mean blood pressure, elevated albumin to creatinine ratio (e.g., >0.6), and hyperglycemia (e.g., blood glucose of over 200 mg/dL). In some embodiments, the subject can have normal kidney function (e.g., GFR=90 mL/min or more). In some embodiments, the subject can have chronic kidney disease (CKD) (e.g., stage 1 CKD (e.g., GFR=90 mL/minute or more)), stage 2 CKD (e.g., GFR=60 to 89 mL/minute), stage 3 CKD (e.g., GFR=30 to 59 mL/minute), stage 4 CKD (e.g., GFR=15 to 29 mL/min), or stage 5 CKD (e.g., GFR=less than 15 mL/min or on dialysis). In some embodiments, the subject has proteinuria (e.g., excretion greater than or equal to 300.mu·g/min albumin). In some embodiments, the subject has CKD (e.g., stage 1, 2, 3, 4, or 5 CKD) and proteinuria.

In some embodiments, the subject has diabetes (e.g., type 1 or type 2 diabetes). In some embodiments, the human subject has type I diabetes (T1D). In some other embodiments, the human subject has type 2 diabetes (T2D).

In one embodiment, methods and compositions described herein can be used to identify therapeutic agents (i.e., reno-protective agents) that may be beneficial for treating or preventing a disorder, disease, or condition associated with progressive kidney function decline. An example of such a condition that may benefit from the predictive methods disclosed herein, includes, but is not limited to, congestive heart failure (Silverberg et al. (2004) Curr Opin Nephrol Hypertens 13(2):163-70).

Kidney function decline, or improvement thereof using the methods disclosed herein, can be measured using eGFR to assess kidney function according to standard practice.

### Kits of the Invention

Further, the present disclosure also provides a kit for performing any of the above-mentioned methods comprising a detectable agent that specifically recognizes the renal associated protein; instructions for use; and optionally, reagents for isolating a sample from the subject. In some embodiments, the said kit determines the level of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more of the renal associated proteins as disclosed herein.

In some embodiments, the kit determines the level of the renal associated protein in a biological sample of a subject, wherein the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof.

The invention is further illustrated by the following example, which should not be construed as limiting. The entire contents of all references, patents and published patent applications cited throughout this application are hereby incorporated by reference in their entirety.

### Example

### Example 1. Application of the Joslin Kidney Panel Using a Proximity Extension Assay: From Prognostics to Precision Medicine

The Joslin Kidney Panel (JKP) of 21 circulating proteins is associated with increased risk of ESKD in patients with diabetes (Kobayashi et al. KI 2022). This study evaluated the JKP as a tool for assessing prognosis in 4 diverse clinical settings.

Concentrations of the JKP proteins were measured in 4 cohorts on a custom OLINK platform using a proximity extension assay. Prognostic value of the JKP proteins for eGFR slope and ESKD during 7-15 years of follow-up was examined in 60 Joslin Kidney Study patients with T1D; for 10-year risk of ESKD in a cohort of 162 Pima Indians with T2D; and for predicting the beneficial effect of fenofibrate in 450 ACCORD trial participants. In addition, the effect of the JAK1/2 inhibitor, Baricitinib, on the 1-year changes of the JKP proteins was examined in 42 Baricitinib trial participants (25 placebo/17 Baricitinib).

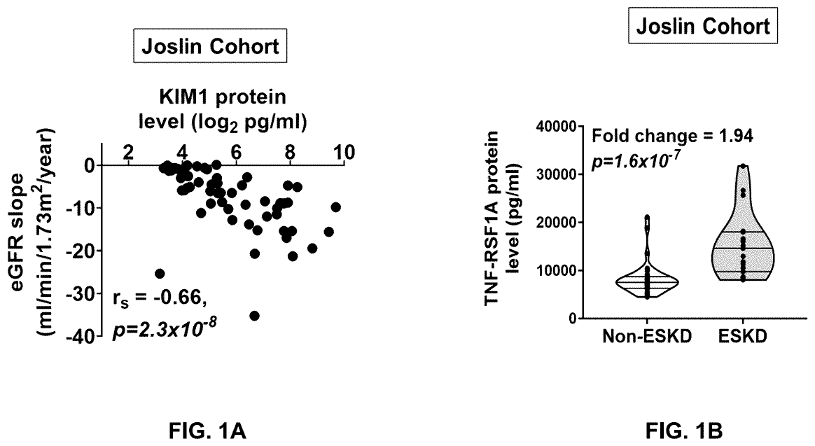

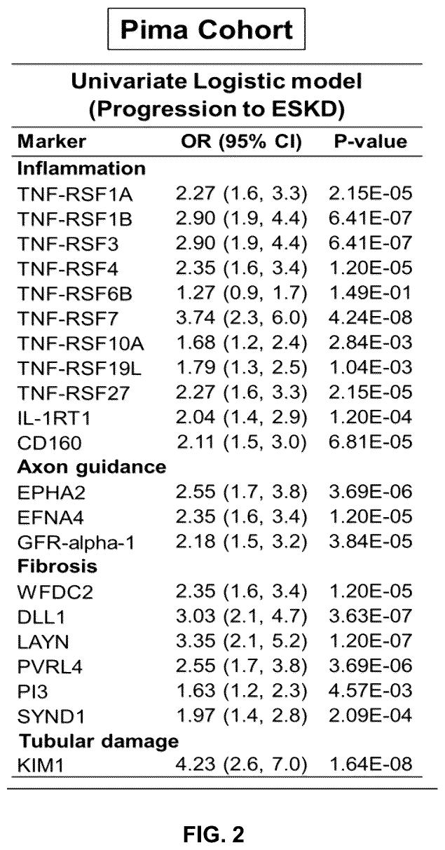

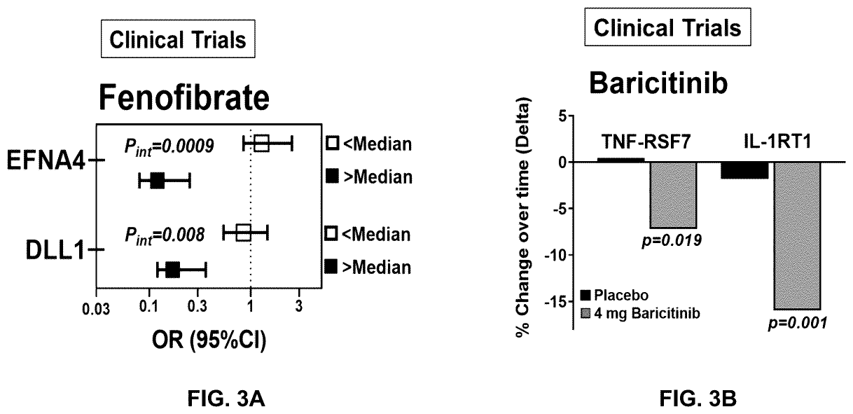

In the Joslin cohort, baseline levels of all proteins correlated strongly with eGFR slope (r=−0.4 to −0.7; p<10⁻⁷) (FIG. 1A for KIM1) and all were significantly higher in patients who developed ESKD than in those who did not (FC=1.3-5.4; p<10⁻⁸) (FIG. 1B for TNFR1). In the Pima cohort, baseline levels of 20 proteins were associated with ESKD risk in univariable logistic model (ORs=1.3-4.2; p<10⁻⁷) (FIG. 2) and the ORs remained significant for 19 proteins (excluding WFDC2 and PI3) after adjustment for key confounders. Fenofibrate treatment reduced loss of kidney function in ACCORD patients who had levels of two proteins above the median (FIG. 3A). Baricitinib significantly decreased levels of 5 proteins relative to placebo (FIG. 3B for TNFR7 and IL-1RT1).

The JKP successfully identified patients at risk of progressive kidney disease and those with beneficial responses to specific reno-protective therapies.

TABLE 1
Sequences

| SEQ ID NO:/ | |
|---|---|
| Description | Sequence |
| 1 | MGLSTVPDLLLPLVLLELLVGIYPSGVIGLVPHLGDREKRDSVCPQ |
| human TNF- | GKYIHPQNNSICCTKCHKGTYLYNDCPGPGQDTDCRECESGSFTA |
| RSF1A | SENHLRHCLSCSKCRKEMGQVEISSCTVDRDTVCGCRKNQYRHY |
| | WSENLFQCFNCSLCLNGTVHLSCQEKQNTVCTCHAGFFLRENECV |
| | SCSNCKKSLECTKLCLPQIENVKGTEDSGTTVLLPLVIFFGLCLLSL |
| | LFIGLMYRYQRWKSKLYSIVCGKSTPEKEGELEGTTTKPLAPNPSF |
| | SPTPGFTPTLGFSPVPSSTFTSSSTYTPGDCPNFAAPRREVAPPYQG |
| | ADPILATALASDPIPNPLQKWEDSAHKPQSLDTDDPATLYAVVEN |
| | VPPLRWKEFVRRLGLSDHEIDRLELQNGRCLREAQYSMLATWRR |
| | RTPRREATLELLGRVLRDMDLLGCLEDIEEALCGPAALPPAPSLLR |
| 2 | MAPVAVWAALAVGLELWAAAHALPAQVAFTPYAPEPGSTCRLR |
| human TNF- | EYYDQTAQMCCSKCSPGQHAKVFCTKTSDTVCDSCEDSTYTQLW |
| RSF1B | NWVPECLSCGSRCSSDQVETQACTREQNRICTCRPGWYCALSKQE |
| | GCRLCAPLRKCRPGFGVARPGTETSDVVCKPCAPGTFSNTTSSTDI |
| | CRPHQICNVVAIPGNASMDAVCTSTSPTRSMAPGAVHLPQPVSTR |
| | SQHTQPTPEPSTAPSTSFLLPMGPSPPAEGSTGDFALPVGLIVGVTA |
| | LGLLIIGVVNCVIMTQVKKKPLCLQREAKVPHLPADKARGTQGPE |
| | QQHLLITAPSSSSSSLESSASALDRRAPTRNQPQAPGVEASGAGEA |
| | RASTGSSDSSPGGHGTQVNVTCIVNVCSSSDHSSQCSSQASSTMG |
| | DTDSSPSESPKDEQVPFSKEECAFRSQLETPETLLGSTEEKPLPLGV |
| | PDAGMKPS |
| 3 | MEATGISLASQLKVPPYASENQTCRDQEKEYYEPQHRICCSRCPPG |
| human TNF- | TYVSAKCSRIRDTVCATCAENSYNEHWNYLTICQLCRPCDPVMGL |
| RSF3 | EEIAPCTSKRKTQCRCQPGMFCAAWALECTHCELLSDCPPGTEAE |
| | LKDEVGKGNNHCVPCKAGHFQNTSSPSARCQPHTRCENQGLVEA |
| | APGTAQSDTTCKNPLEPLPPEMSGTMLMLAVLLPLAFFLLLATVFS |
| | CIWKSHPSLCRKLGSLLKRRPQGEGPNPVAGSWEPPKAHPYFPDL |
| | VQPLLPISGDVSPVSTGLPAAPVLEAGVPQQQSPLDLTREPQLEPG |
| | EQSQVAHGTNGIHVTGGSMTITGNIYIYNGPVLGGPPGPGDLPATP |
| | EPPYPIPEEGDPGPPGLSTPHQEDGKAWHLAETEHCGATPSNRGPR |
| | NQFITHD |
| 4 | MCVGARRLGRGPCAALLLLGLGLSTVTGLHCVGDTYPSNDRCCH |
| human TNF- | ECRPGNGMVSRCSRSQNTVCRPCGPGFYNDVVSSKPCKPCTWCN |
| RSF4 | LRSGSERKQLCTATQDTVCRCRAGTQPLDSYKPGVDCAPCPPGHF |
| | SPGDNQACKPWTNCTLAGKHTLQPASNSSDAICEDRDPPATQPQE |
| | TQGPPARPITVQPTEAWPRTSQGPSTRPVEVPGGRAVAAILGLGLV |
| | LGLLGPLAILLALYLLRRDQRLPPDAHKPPGGGSFRTPIQEEQADA |
| | HSTLAKI |
| 5 | MRALEGPGLSLLCLVLALPALLPVPAVRGVAETPTYPWRDAETGE |
| human TNF- | RLVCAQCPPGTFVQRPCRRDSPTTCGPCPPRHYTQFWNYLERCRY |
| RSF6B | CNVLCGEREEEARACHATHNRACRCRTGFFAHAGFCLEHASCPP |
| | GAGVIAPGTPSQNTQCQPCPPGTFSASSSSSEQCQPHRNCTALGLA |
| | LNVPGSSSHDTLCTSCTGFPLSTRVPGAEECERAVIDFVAFQDISIK |
| | RLQRLLQALEAPEGWGPTPRAGRAALQLKLRRRLTELLGAQDGA |
| | LLVRLLQALRVARMPGLERSVRERFLPVH |
| 6 | MARPHPWWLCVLGTLVGLSATPAPKSCPERHYWAQGKLCCQMC |
| human TNF- | EPGTFLVKDCDQHRKAAQCDPCIPGVSFSPDHHTRPHCESCRHCN |
| RSF7 | SGLLVRNCTITANAECACRNGWQCRDKECTECDPLPNPSLTARSS |
| | QALSPHPQPTHLPYVSEMLEARTAGHMQTLADFRQLPARTLSTH |
| | WPPQRSLCSSDFIRILVIFSGMFLVFTLAGALFLHQRRKYRSNKGES |
| | PVEPAEPCHYSCPREEEGSTIPIQEDYRKPEPACSP |
| 7 | MAPPPARVHLGAFLAVTPNPGSAASGTEAAAATPSKVWGSSAGRI |
| human TNF- | EPRGGGRGALPTSMGQHGPSARARAGRAPGPRPAREASPRLRVH |
| RSF10A | KTFKFVVVGVLLQVVPSSAATIKLHDQSIGTQQWEHSPLGELCPP |
| | GSHRSEHPGACNRCTEGVGYTNASNNLFACLPCTACKSDEEERSP |
| | CTTTRNTACQCKPGTFRNDNSAEMCRKCSRGCPRGMVKVKDCTP |
| | WSDIECVHKESGNGHNIWVILVVTLVVPLLLVAVLIVCCCIGSGCG |
| | GDPKCMDRVCFWRLGLLRGPGAEDNAHNEILSNADSLSTFVSEQ |
| | QMESQEPADLTGVTVQSPGEAQCLLGPAEAEGSQRRRLLVPANG |
| | ADPTETLMLFFDKFANIVPFDSWDQLMRQLDLTKNEIDVVRAGTA |
| | GPGDALYAMLMKWVNKTGRNASIHTLLDALERMEERHAREKIQ |
| | DLLVDSGKFIYLEDGTGSAVSLE |
| 8 | MEQRGQNAPAASGARKRHGPGPREARGARPGPRVPKTLVLVVA |
| human TNF- | AVLLLVSAESALITQQDLAPQQRAAPQQKRSSPSEGLCPPGHHISE |
| RSF10B | DGRDCISCKYGQDYSTHWNDLLFCLRCTRCDSGEVELSPCTTTRN |
| | TVCQCEEGTFREEDSPEMCRKCRTGCPRGMVKVGDCTPWSDIEC |
| | VHKESGTKHSGEVPAVEETVTSSPGTPASPCSLSGIIIGVTVAAVVL |
| | IVAVFVCKSLLWKKVLPYLKGICSGGGGDPERVDRSSQRPGAEDN |
| | VLNEIVSILQPTQVPEQEMEVQEPAEPTGVNMLSPGESEHLLEPAE |
| | AERSQRRRLLVPANEGDPTETLRQCFDDFADLVPFDSWEPLMRKL |
| | GLMDNEIKVAKAEAAGHRDTLYTMLIKWVNKTGRDASVHTLLD |
| | ALETLGERLAKQKIEDHLLSSGKFMYLEGNADSAMS |
| 9 | MAPRARRRRPLFALLLLCALLARLQVALQIAPPCTSEKHYEHLGR |
| TNF-RSF11A | CCNKCEPGKYMSSKCTTTSDSVCLPCGPDEYLDSWNEEDKCLLH |
| | KVCDTGKALVAVVAGNSTTPRRCACTAGYHWSQDCECCRRNTE |
| | CAPGLGAQHPLQLNKDTVCKPCLAGYFSDAFSSTDKCRPWTNCT |
| | FLGKRVEHHGTEKSDAVCSSSLPARKPPNEPHVYLPGLIILLLFASV |
| | ALVAAIIFGVCYRKKGKALTANLWHWINEACGRLSGDKEM |
| 10 | MKPSLLCRPLSCFLMLLPWPLATLTSTTLWQCPPGEEPDLDPGQG |
| human TNF- | TLCRPCPPGTFSAAWGSSPCQPHARCSLWRRLEAQVGMATRDTL |
| RSF19L | CGDCWPGWFGPWGVPRVPCQPCSWAPLGTHGCDEWGRRARRG |
| | VEVAAGASSGGETRQPGNGTRAGGPEETAAQYAVIAIVPVFCLM |
| | GLLGILVCNLLKRKGYHCTAHKEVGPGPGGGGSGINPAYRTEDA |
| | NEDTIGVLVRLITEKKENAAALEELLKEYHSKQLVQTSHRPVSKLP |
| | PAPPNVPHICPHRHHLHTVQGLASLSGPCCSRCSQKKWPEVLLSPE |
| | AVAATTPVPSLLPNPTRVPKAGAKAGRQGEITILSVGRFRVARIPE |
| | QRTSSMVSEVKTITEAGPSWGDLPDSPQPGLPPEQQALLGSGGSRT |
| | KWLKPPAENKAEENRYVVRLSESNLVI |
| 11 | MDCQENEYWDQWGRCVTCQRCGPGQELSKDCGYGEGGDAYCT |
| human TNF- | ACPPRRYKSSWGHHRCQSCITCAVINRVQKVNCTATSNAVCGDC |
| RSF27 | LPRFYRKTRIGGLQDQECIPCTKQTPTSEVQCAFQLSLVEADTPTV |
| | PPQEATLVALVSSLLVVFTLAFLGLFFLYCKQFFNRHCQRGGLLQF |
| | EADKTAKEESLFPVPPSKETSAESQVSENIFQTQPLNPILEDDCSSTS |
| | GFPTQESFTMASCTSESHSHWVHSPIECTELDLQKFSSSASYTGAE |
| | TLGGNTVESTGDRLELNVPFEVPSP |
| 12 | MKVLLRLICFIALLISSLEADKCKEREEKIILVSSANEIDVRPCPLNP |
| human IL-1RT1 | NEHKGTITWYKDDSKTPVSTEQASRIHQHKEKLWFVPAKVEDSG |
| | HYYCVVRNSSYCLRIKISAKFVENEPNLCYNAQAIFKQKLPVAGD |
| | GGLVCPYMEFFKNENNELPKLQWYKDCKPLLLDNIHFSGVKDRLI |
| | VMNVAEKHRGNYTCHASYTYLGKQYPITRVIEFITLEENKPTRPVI |
| | VSPANETMEVDLGSQIQLICNVTGQLSDIAYWKWNGSVIDEDDPV |
| | LGEDYYSVENPANKRRSTLITVLNISEIESRFYKHPFTCFAKNTHGI |
| | DAAYIQLIYPVTNFQKHMIGICVTLTVIIVCSVFIYKIFKIDIVLWYR |
| | DSCYDFLPIKASDGKTYDAYILYPKTVGEGSTSDCDIFVFKVLPEV |
| | LEKQCGYKLFIYGRDDYVGEDIVEVINENVKKSRRLIIILVRETSGF |
| | SWLGGSSEEQIAMYNALVQDGIKVVLLELEKIQDYEKMPESIKFIK |
| | QKHGAIRWSGDFTQGPQSAKTRFWKNVRYHMPVQRRSPSSKHQL |
| | LSPATKEKLQREAHVPLG |
| 13 | MLLEPGRGCCALAILLAIVDIQSGGCINITSSASQEGTRLNLICTVW |
| human CD160 | HKKEEAEGFVVFLCKDRSGDCSPETSLKQLRLKRDPGIDGVGEISS |
| | QLMFTISQVTPLHSGTYQCCARSQKSGIRLQGHFFSILFTETGNYT |
| | VTGLKQRQHLEFSHNEGTLSSGFLQEKVWVMLVTSLVALQAL |
| 14 | MQNIMNDMPIYMYSVCNVMSGDQDNWLRTNWVYRGEAERIFIE |
| human EPHA2 | LKFTVRDCNSFPGGASSCKETFNLYYAESDLDYGTNFQKRLFTKI |
| | DTIAPDEITVSSDFEARHVKLNVEERSVGPLTRKGFYLAFQDIGAC |
| | VALLSVRVYYKKCPELLQGLAHFPETIAGSDAPSLATVAGTCVDH |
| | AVVPPGGEEPRMHCAVDGEWLVPIGQCLCQAGYEKVEDACQAC |
| | SPGFFKFEASESPCLECPEHTLPSPEGATSCECEEGFFRAPQDPASM |
| | PCTRPPSAPHYLTAVGMGAKVELRWTPPQDSGGREDIVYSVTCEQ |
| | CWPESGECGPCEASVRYSEPPHGLTRTSVTVSDLEPHMNYTFTVE |
| | ARNGVSGLVTSRSFRTASVSINQTEPPKVRLEGRSTTSLSVSWSIPP |
| | PQQSRVWKYEVTYRKKGDSNSYNVRRTEGFSVTLDDLAPDTTYL |
| | VQVQALTQEGQGAGSKVHEFQTLSPEGSGNLAVIGGVAVGVVLL |
| | LVLAGVGFFIHRRRKNQRARQSPEDVYFSKSEQLKPLKTYVDPHT |
| | YEDPNQAVLKFTTEIHPSCVTRQKVIGAGEFGEVYKGMLKTSSGK |
| | KEVPVAIKTLKAGYTEKQRVDFLGEAGIMGQFSHHNIIRLEGVISK |
| | YKPMMIITEYMENGALDKFLREKDGEFSVLQLVGMLRGIAAGMK |
| | YLANMNYVHRDLAARNILVNSNLVCKVSDFGLSRVLEDDPEATY |
| | TTSGGKIPIRWTAPEAISYRKFTSASDVWSFGIVMWEVMTYGERP |
| | YWELSNHEVMKAINDGFRLPTPMDCPSAIYQLMMQCWQQERAR |
| | RPKFADIVSILDKLIRAPDSLKTLADFDPRVSIRLPSTSGSEGVPFRT |
| | VSEWLESIKMQQYTEHFMAAGYTAIEKVVQMTNDDIKRIGVRLP |
| | GHQKRIAYSLLGLKDQVNTVGIPI |
| 15 | MRLLPLLRTVLWAAFLGSPLRGGSSLRHVVYWNSSNPRLLRGDA |
| human EFNA4 | VVELGLNDYLDIVCPHYEGPGPPEGPETFALYMVDWPGYESCQA |
| | EGPRAYKRWVCSLPFGHVQFSEKIQRFTPFSLGFEFLPGETYYYIS |
| | VPTPESSGQCLRLQVSVCCKERKSESAHPVGSPGESGTSGWRGGD |
| | TPSPLCLLLLLLLLILRLLRIL |
| 16 | MFLATLYFALPLLDLLLSAEVSGGDRLDCVKASDQCLKEQSCSTK |
| human GFR- | YRTLRQCVAGKETNFSLASGLEAKDECRSAMEALKQKSLYNCRC |
| alpha-1 | KRGMKKEKNCLRIYWSMYQSLQGNDLLEDSPYEPVNSRLSDIFRV |
| | VPFISVEHIPKGNNCLDAAKACNLDDICKKYRSAYITPCTTSVSND |
| | VCNRRKCHKALRQFFDKVPAKHSYGMLFCSCRDIACTERRRQTIV |
| | PVCSYEEREKPNCLNLQDSCKTNYICRSRLADFFTNCQPESRSVSS |
| | CLKENYADCLLAYSGLIGTVMTPNYIDSSSLSVAPWCDCSNSGND |
| | LEECLKFLNFFKDNTCLKNAIQAFGNGSDVTVWQPAFPVQTTTAT |
| | TTTALRVKNKPLGPAGSENEIPTHVLPPCANLQAQKLKSNVSGNT |
| | HLCISNGNYEKEGLGASSHITTKSMAAPPSCGLSPLLVLVVTALST |
| | LLSLTETS |
| 17 | MPACRLGPLAAALLLSLLLFGFTLVSGTGAEKTGVCPELQADQNC |
| human WFDC2 | TQECVSDSECADNLKCCSAGCATFCSLPNDKEGSCPQVNINFPQL |
| | GLCRDQCQVDSQCPGQMKCCRNGCGKVSCVTPNF |
| 18 | MGSRCALALAVLSALLCQVWSSGVFELKLQEFVNKKGLLGNRNC |
| human DLL1 | CRGGAGPPPCACRTFFRVCLKHYQASVSPEPPCTYGSAVTPVLGV |
| | DSFSLPDGGGADSAFSNPIRFPFGFTWPGTFSLIIEALHTDSPDDLA |
| | TENPERLISRLATQRHLTVGEEWSQDLHSSGRTDLKYSYRFVCDE |
| | HYYGEGCSVFCRPRDDAFGHFTCGERGEKVCNPGWKGPYCTEPI |
| | CLPGCDEQHGFCDKPGECKCRVGWQGRYCDECIRYPGCLHGTCQ |
| | QPWQCNCQEGWGGLFCNQDLNYCTHHKPCKNGATCTNTGQGSY |
| | TCSCRPGYTGATCELGIDECDPSPCKNGGSCTDLENSYSCTCPPGF |
| | YGKICELSAMTCADGPCFNGGRCSDSPDGGYSCRCPVGYSGFNCE |
| | KKIDYCSSSPCSNGAKCVDLGDAYLCRCQAGFSGRHCDDNVDDC |
| | ASSPCANGGTCRDGVNDFSCTCPPGYTGRNCSAPVSRCEHAPCHN |
| | GATCHERGHRYVCECARGYGGPNCQFLLPELPPGPAVVDLTEKLE |
| | GQGGPFPWVAVCAGVILVLMLLLGCAAVVVCVRLRLQKHRPPA |
| | DPCRGETETMNNLANCQREKDISVSIIGATQIKNTNKKADFHGDH |
| | SADKNGFKARYPAVDYNLVQDLKGDDTAVRDAHSKRDTKCQPQ |
| | GSSGEEKGTPTTLRGGEASERKRPDSGCSTSKDTKYQSVYVISEEK |
| | DECVIATEV |
| 19 | MRPGTALQAVLLAVLLVGLRAATGRLLSASDLDLRGGQPVCRGG |
| human LAYN | TQRPCYKVIYFHDTSRRLNFEEAKEACRRDGGQLVSIESEDEQKLI |
| | EKFIENLLPSDGDFWIGLRRREEKQSNSTACQDLYAWTDGSISQFR |
| | NWYVDEPSCGSEVCVVMYHQPSAPAGIGGPYMFQWNDDRCNM |
| | KNNFICKYSDEKPAVPSREAEGEETELTTPVLPEETQEEDAKKTFK |
| | ESREAALNLAYILIPSIPLLLLLVVTTVVCWVWICRKRKREQPDPST |
| | KKQHTIWPSPHQGNSPDLEVYNVIRKQSEADLAETRPDLKNISFRV |
| | CSGEATPDDMSCDYDNMAVNPSESGFVTLVSVESGFVTNDIYEFS |
| | PDQMGRSKESGWVENEIYGY |
| 20 | MPLSLGAEMWGPEAWLLLLLLLASFTGRCPAGELETSDVVTVVL |
| human PVRL4 | GQDAKLPCFYRGDSGEQVGQVAWARVDAGEGAQELALLHSKYG |
| | LHVSPAYEGRVEQPPPPRNPLDGSVLLRNAVQADEGEYECRVSTF |
| | PAGSFQARLRLRVLVPPLPSLNPGPALEEGQGLTLAASCTAEGSPA |
| | PSVTWDTEVKGTTSSRSFKHSRSAAVTSEFHLVPSRSMNGQPLTC |
| | VVSHPGLLQDQRITHILHVSFLAEASVRGLEDQNLWHIGREGAML |
| | KCLSEGQPPPSYNWTRLDGPLPSGVRVDGDTLGFPPLTTEHSGIYV |
| | CHVSNEFSSRDSQVTVDVLDPQEDSGKQVDLVSASVVVVGVIAA |
| | LLFCLLVVVVVLMSRYHRRKAQQMTQKYEEELTLTRENSIRRLHS |
| | HHTDPRSQPEESVGLRAEGHPDSLKDNSSCSVMSEEPEGRSYSTLT |
| | TVREIETQTELLSPGSGRAEEEEDQDEGIKQAMNHFVQENGTLRA |
| | KPTGNGIYINGRGHLV |
| 21 | MRASSFLIVVVFLIAGTLVLEAAVTGVPVKGQDTVKGRVPFNGQD |
| human PI3 | PVKGQVSVKGQDKVKAQEPVKGPVSTKPGSCPIILIRCAMLNPPN |
| | RCLKDTDCPGIKKCCEGSCGMACFVPQ |
| 22 | MRRAALWLWLCALALSLQPALPQIVATNLPPEDQDGSGDDSDNF |
| human SYND1 | SGSGAGALQDITLSQQTPSTWKDTQLLTAIPTSPEPTGLEATAASTS |
| | TLPAGEGPKEGEAVVLPEVEPGLTAREQEATPRPRETTQLPTTHLA |
| | STTTATTAQEPATSHPHRDMQPGHHETSTPAGPSQADLHTPHTED |
| | GGPSATERAAEDGASSQLPAAEGSGEQDFTFETSGENTAVVAVEP |
| | DRRNQSPVDQGATGASQGLLDRKEVLGGVIAGGLVGLIFAVCLV |
| | GFMLYRMKKKDEGSYSLEEPKQANGGAYQKPTKQEEFYA |
| 23 | MHPQVVILSLILHLADSVAGSVKVGGEAGPSVTLPCHYSGAVTSM |
| human KIM1 | CWNRGSCSLFTCQNGIVWTNGTHVTYRKDTRYKLLGDLSRRDVS |
| | LTIENTAVSDSGVYCCRVEHRGWFNDMKITVSLEIVPPKVTTTPIV |
| | TTVPTVTTVRTSTTVPTTTTVPMTTVPTTTVPTTMSIPTTTTVLTTM |
| | TVSTTTSVPTTTSIPTTTSVPVTTTVSTFVPPMPLPRQNHEPVATSPS |
| | SPQPAETHPTTLQGAIRREPTSSPLYSYTTDGNDTVTESSDGLWNN |
| | NQTQLFLEHSLLTANTTKGIYAGVCISVLVLLALLGVIIAKKYFFK |
| | KEVQQLSVSFSSLQIKALQNAVEKEVQAEDNIYIENSLYATD |
| 24 | MDLWNLSWFLFLDALLVISGLATPENFDVDGGMDQDIFDINEGLG |
| human MEP1B | LDLFEGDIRLDRAQIRNSIIGEKYRWPHTIPYVLEDSLEMNAKGVIL |
| | NAFERYRLKTCIDFKPWAGETNYISVFKGSGCWSSVGNRRVGKQ |
| | ELSIGANCDRIATVQHEFLHALGFWHEQSRSDRDDYVRIMWDRIL |
| | SGREHNFNTYSDDISDSLNVPYDYTSVMHYSKTAFQNGTEPTIVT |
| | RISDFEDVIGQRMDFSDSDLLKLNQLYNCSSSLSFMDSCSFELENV |
| | CGMIQSSGDNADWQRVSQVPRGPESDHSNMGQCQGSGFFMHFDS |
| | SSVNVGATAVLESRTLYPKRGFQCLQFYLYNSGSESDQLNIYIREY |
| | SADNVDGNLTLVEEIKEIPTGSWQLYHVTLKVTKKFRVVFEGRKG |
| | SGASLGGLSIDDINLSETRCPHHIWHIRNFTQFIGSPNGTLYSPPFYS |
| | SKGYAFQIYLNLAHVTNAGIYFHLISGANDDQLQWPCPWQQATM |
| | TLLDQNPDIRQRMSNQRSITTDPFMTTDNGNYFWDRPSKVGTVAL |
| | FSNGTQFRRGGGYGTSAFITHERLKSRDFIKGDDVYILLTVEDISHL |
| | NSTQIQLTPAPSVQDLCSKTTCKNDGVCTVRDGKAECRCQSGED |
| | WWYMGERCEKRGSTRDTIVIAVSSTVAVFALMLIITLVSVYCTRK |
| | KYRERMSSNRPNLTPQNHAF |
| 25 | MGRPLLLPLLLLLQPPAFLQPGGSTGSGPSYLYGVTQPKHLSASM |
| human PILRB | GGSVEIPFSFYYPWELAIVPNVRISWRRGHFHGQSFYSTRPPSIHKD |
| | YVNRLFLNWTEGQESGFLRISNLRKEDQSVYFCRVELDTRRSGRQ |
| | QLQSIKGTKLTITQAVTTTTTWRPSSTTTIAGLRVTESKGHSESWH |
| | LSLDTAIRVALAVAVLKTVILGLLCLLLLWWRRRKGLSHRTRPV |
| | WNLLYGRSMCGISAELSIGNLDVHISSSKERTRLQAAMSSTSISSKP |
| | DSSRAPSSDF |
| 26 | MPGQELRTVNGSQMLLVLLVLSWLPHGGALSLAEASRASFPGPSE |
| human GDF15 | LHSEDSRFRELRKRYEDLLTRLRANQSWEDSNTDLVPAPAVRILTP |
| | EVRLGSGGHLHLRISRAALPEGLPEASRLHRALFRLSPTASRSWDV |
| | TRPLRRQLSLARPQAPALHLRLSPPPSQSDQLLAESSSARPQLELHL |
| | RPQAARGRRRARARNGDHCPLGPGRCCRLHTVRASLEDLGWAD |
| | WVLSPREVQVTMCIGACPSQFRAANMHAQIKTSLHRLKPDTVPAP |
| | CCVPASYNPMVLIQKTDTGVSLQTYDDLLAKDCHCI |
| 27 | MTVFLSFAFLAAILTHIGCSNQRRSPENSGRRYNRIQHGQCAYTFIL |
| human ANGPT1 | PEHDGNCRESTTDQYNTNALQRDAPHVEPDFSSQKLQHLEHVME |
| | NYTQWLQKLENYIVENMKSEMAQIQQNAVQNHTATMLEIGTSLL |
| | SQTAEQTRKLTDVETQVLNQTSRLEIQLLENSLSTYKLEKQLLQQT |
| | NEILKIHEKNSLLEHKILEMEGKHKEELDTLKEEKENLQGLVTRQT |
| | YIIQELEKQLNRATTNNSVLQKQQLELMDTVHNLVNLCTKEGVLL |
| | KGGKREEEKPFRDCADVYQAGFNKSGIYTIYINNMPEPKKVFCNM |
| | DVNGGGWTVIQHREDGSLDFQRGWKEYKMGFGNPSGEYWLGNE |
| | FIFAITSQRQYMLRIELMDWEGNRAYSQYDRFHIGNEKQNYRLYL |
| | KGHTGTAGKQSSLILHGADFSTKDADNDNCMCKCALMLTGGWW |
| | FDACGPSNLNGMFYTAGQNHGKLNGIKWHYFKGPSYSLRSTTM |
| | MIRPLDF |
| 28 | MWQIVFFTLSCDLVLAAAYNNFRKSMDSIGKKQYQVQHGSCSYT |
| human ANGPT2 | FLLPEMDNCRSSSSPYVSNAVQRDAPLEYDDSVQRLQVLENIMEN |
| | NTQWLMKLENYIQDNMKKEMVEIQQNAVQNQTAVMIEIGTNLL |
| | NQTAEQTRKLTDVEAQVLNQTTRLELQLLEHSLSTNKLEKQILDQ |
| | TSEINKLQDKNSFLEKKVLAMEDKHIIQLQSIKEEKDQLQVLVSKQ |
| | NSIIEELEKKIVTATVNNSVLQKQQHDLMETVNNLLTMMSTSNSK |
| | DPTVAKEEQISFRDCAEVFKSGHTTNGIYTLTFPNSTEEIKAYCDM |
| | EAGGGGWTIIQRREDGSVDFQRTWKEYKVGFGNPSGEYWLGNEF |
| | VSQLTNQQRYVLKIHLKDWEGNEAYSLYEHFYLSSEELNYRIHLK |
| | GLTGTAGKISSISQPGNDFSTKDGDNDKCICKCSQMLTGGWWFDA |
| | CGPSNLNGMYYPQRQNTNKFNGIKWYYWKGSGYSLKATTMMIR |
| | PADF |
| 29 | MAARRSQRRRGRRGEPGTALLVPLALGLGLALACLGLLLAVVSL |
| human TNFSF12 | GSRASLSAQEPAQEELVAEEDQDPSELNPQTEESQDPAPFLNRLVR |
| | PRRSAPKGRKTRARRAIAAHYEVHPRPGQDGAQAGVDGTVSGWE |
| | EARINSSSPLRYNRQIGEFIVTRAGLYYLYCQVHFDEGKAVYLKLD |
| | LLVDGVLALRCLEEFSATAASSLGPQLRLCQVSGLLALRPGSSLRI |
| | RTLPWAHLKAAPFLTYFGLFQVH |
| 30 | MASVAQESAGSQRRLPPRHGALRGLLLLCLWLPSGRAALPPAAPL |
| human LRP11 | SELHAQLSGVEQLLEEFRRQLQQERPQEELELELRAGGGPQEDCP |
| | GPGSGGYSAMPDAIIRTKDSLAAGASFLRAPAAVRGWRQCVAAC |
| | CSEPRCSVAVVELPRRPAPPAAVLGCYLFNCTARGRNVCKFALHS |
| | GYSSYSLSRAPDGAALATARASPRQEKDAPPLSKAGQDVVLHLPT |
| | DGVVLDGRESTDDHAIVQYEWALLQGDPSVDMKVPQSGTLKLSH |
| | LQEGTYTFQLTVTDTAGQRSSDNVSVTVLRAAYSTGGCLHTCSRY |
| | HFFCDDGCCIDITLACDGVQQCPDGSDEDFCQNLGLDRKMVTHT |
| | AASPALPRTTGPSEDAGGDSLVEKSQKATAPNKPPALSNTEKRNH |
| | SAFWGPESQIIPVMPDSSSSGKNRKEESYIFESKGDGGGGEHPAPE |
| | TGAVLPLALGLAITALLLLMVACRLRLVKQKLKKARPITSEESDY |
| | LINGMYL |
| 31 | MRAPGCGRLVLPLLLLAAAALAEGDAKGLKEGETPGNFMEDEQ |
| human | WLSSISQYSGKIKHWNRFRDEVEDDYIKSWEDNQQGDEALDTTK |
| Testican-2 | DPCQKVKCSRHKVCIAQGYQRAMCISRKKLEHRIKQPTVKLHGN |
| | KDSICKPCHMAQLASVCGSDGHTYSSVCKLEQQACLSSKQLAVR |
| | CEGPCPCPTEQAATSTADGKPETCTGQDLADLGDRLRDWFQLLH |
| | ENSKQNGSASSVAGPASGLDKSLGASCKDSIGWMFSKLDTSADLF |
| | LDQTELAAINLDKYEVCIRPFFNSCDTYKDGRVSTAEWCFCFWRE |
| | KPPCLAELERIQIQEAAKKKPGIFIPSCDEDGYYRKMQCDQSSGDC |
| | WCVDQLGLELTGTRTHGSPDCDDIVGFSGDFGSGVGWEDEEEKE |
| | TEEAGEEAEEEEGEAGEADDGGYIW |
| 32 | MEDYPDPQSANHMNSSLLSLYRKGNPDSVSNTPEMEQRETTSSTP |
| human NVL1 | RISSKTGSIPLKTPAKDSEGGWFIDKTPSVKKDSFFLDLSCEKSNPK |
| | KPITEIQDSKDSSLLESDMKRKGKLKNKGSKRKKEDLQEVDGEIE |
| | AVLQKKAKARGLEFQISNVKFEDVGGNDMTLKEVCKMLIHMRHP |
| | EVYHHLGVVPPRGVLLHGPPGCGKTLLAHAIAGELDLPILKVAAP |
| | EIVSGVSGESEQKLRELFEQAVSNAPCIIFIDEIDAITPKREVASKDM |
| | ERRIVAQLLTCMDDLNNVAATARVLVIGATNRPDSLDPALRRAG |
| | RFDREICLGIPDEASRERILQTLCRKLRLPQAFDFCHLAHLTPGFVG |
| | ADLMALCREAAMCAVNRVLMKLQEQQKKNPEMEDLPSKGVQEE |
| | RLGTEPTSETQDELQRLLGLLRDQDPLSEEQMQGLCIELNDFIVAL |
| | SSVQPSAKREGFVTVPNVTWADIGALEDIREELTMAILAPVRNPD |
| | QFKALGLVTPAGVLLAGPPGCGKTLLAKAVANESGLNFISVKGPE |
| | LLNMYVGESERAVRQVFQRAKNSAPCVIFFDEVDALCPRRSDRET |
| | GASVRVVNQLLTEMDGLEARQQVFIMAATNRPDIIDPAILRPGRL |
| | DKTLFVGLPPPADRLAILKTITKNGTKPPLDADVNLEAIAGDLRCD |
| | CYTGADLSALVREASICALRQEMARQKSGNEKGELKVSHKHFEE |
| | AFKKVRSSISKKDQIMYERLQESLSR |

1. A method for determining whether a human subject will respond to a reno-protective agent for the treatment or prevention of progressive kidney function decline, said method comprising
detecting the level of a renal associated protein in a biological sample from a human subject having or at risk of having progressive kidney function decline, wherein the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof, and
comparing the level of the renal associated protein with a responder control level;
wherein the human subject is a responder to the reno-protective agent if the level of the renal associated protein is equal to or higher than the responder control level, and wherein the human subject is not a responder to the reno-protective agent if the level of the renal associated protein is less than the responder control level.

2. A method for determining whether a human subject will respond to a reno-protective agent for the treatment or prevention of progressive kidney function decline, said method comprising
detecting the level of a renal associated protein in a biological sample from a human subject having or at risk of having progressive kidney function decline, wherein the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof, and
comparing the level of the renal associated protein with a non-responder control level;
wherein the human subject is a non-responder to the reno-protective agent if the level of the renal associated protein is equal to or higher than the non-responder control level, and wherein the human subject is not a non-responder to the reno-protective agent if the level of the renal associated protein is less than the non-responder control level.

3. The method of claim 1, wherein the reno-protective agent is fenofibrate, baricitinib, SGLT2 inhibitor, or a GLP-1/GIP agonist.

4-6. (canceled)

7. The method of claim 1, wherein the protein level is determined by an assay selected from the group consisting of an immunoassay, a mass spectrometry analysis, a Slow Off-rate Modified Aptamer (SOMA) scan platform analysis, liquid chromatography (LC) fractionation, Mesoscale platform, electrochemiluminescence detection, or an OLINK Proximity Extension Assay based proteomic platform analysis.

8. The method of claim 1, wherein the kidney function decline is progression from normal kidney function to chronic kidney disease, or from chronic kidney disease to end stage kidney disease (ESKD).

9. (canceled)

10. The method of claim 1, wherein the human subject has at least one of type 1 diabetes (T1D), type 2 diabetes (T2D), diabetic kidney disease, cystinosis, glomerulonephritis, polycystic kidney disease, IgA nephropathy, early progressive renal decline, or late progressive renal decline.

11-15. (canceled)

16. A method for treating or preventing progressive kidney function decline in a human subject, the method comprising
determining whether the human subject will respond to a reno-protective agent for the treatment or prevention of progressive kidney function decline, comprising the steps of:
detecting the level of a renal associated protein in a biological sample from the human subject, wherein the renal associated protein is TNF-RSF1A, TNF-RSF1B, TNF-RSF3, TNF-RSF4, TNF-RSF6B, TNF-RSF7, TNF-RSF10A, TNF-RSF10B, TNF-RSF11A, TNF-RSF19L, TNF-RSF27, IL-1RT1, CD160, EPHA2, EFNA4, GFR-alpha-1, WFDC2, DLL1, LAYN, PVRL4, PI3, SYND1, KIM1, MEP1B, PILRB, GDF15, ANGPT1, ANGPT2, TNFSF12, LRP11, Testican, NVL1, or a combination thereof, and
comparing the level of the renal associated protein with a responder control level;
wherein the human subject is a responder to the reno-protective agent if the level of the renal associated protein is equal to or higher than a responder control level, and wherein the human subject is not a responder to the reno-protective agent if the level of the renal associated protein is less than the responder control level; and
administering the reno-protective agent to the responder, such that the progressive kidney function decline is treated or prevented.

17. The method of claim 16, wherein the reno-protective agent is fenofibrate, baricitinib, SGLT2 inhibitor, or a GLP-1/GIP agonist.

18-20. (canceled)

21. The method of claim 16, wherein the protein level is determined using an assay selected from the group consisting of an immunoassay, a mass spectrometry analysis, a Slow Off-rate Modified Aptamer (SOMA) scan platform analysis, liquid chromatography (LC) fractionation, Mesoscale platform, electrochemiluminescence detection, or an OLINK Proximity Extension Assay based proteomic platform analysis.

22. The method of claim 16, wherein the kidney function decline is progression from normal kidney function to chronic kidney disease, or from chronic kidney disease to end stage kidney disease (ESKD).

23. (canceled)

24. The method of claim 16, wherein the human subject has at least one of type 1 diabetes (T1D), type 2 diabetes (T2D), diabetic kidney disease, cystinosis, glomerulonephritis, polycystic kidney disease, IgA nephropathy, early progressive renal decline, or late progressive renal decline.

25-29. (canceled)

30. A method for determining whether a human subject will respond to fenofibrate for the treatment or prevention of progressive kidney function decline, said method comprising
detecting the level of a renal associated protein in a biological sample from the human subject, wherein the renal associated protein is EFNA4, DLL1, or a combination thereof, and
comparing the level of the renal associated protein with a responder control level;
wherein the human subject is a responder to fenofibrate if the level of the renal associated protein is equal to or higher than a responder control level, and wherein the human subject is not a responder to fenofibrate if the level of the renal associated protein is less than the responder control level.

31. The method of claim 30, further comprising administering an effective amount of fenofibrate to the responder such that the progressive kidney function decline is treated.

32. A method for determining whether a human subject will respond to baricitinib for the treatment or prevention of progressive kidney function decline, said method comprising
detecting the level of a renal associated protein in a biological sample from the human subject, wherein the renal associated protein is TNF-RSF7, IL-1RT1, or a combination thereof, and
comparing the level of the renal associated protein with a responder control level;
wherein the human subject is a responder to baricitinib if the level of the renal associated protein is equal to or higher than a responder control level, and wherein the human subject is not a responder to baricitinib if the level of the renal associated protein is less than the responder control level.

33. The method of claim 32, further comprising administering an effective amount of baricitinib to the responder such that the progressive kidney function decline is treated.

34. A method for determining whether a human subject will respond to SGL2 for the treatment or prevention of progressive kidney function decline, said method comprising
detecting the level of a renal associated protein in a biological sample from the human subject and
comparing the level of the renal associated protein with a responder control level;
wherein the human subject is a responder to SGL2 if the level of the renal associated protein is equal to or higher than a responder control level, and wherein the human subject is not a responder to SGL2 if the level of the renal associated protein is less than the responder control level.

35. The method of claim 34, further comprising administering an effective amount of SGL2 to the responder such that the progressive kidney function decline is treated.

36. The method of claim 2, wherein the protein level is determined by an assay selected from the group consisting of an immunoassay, a mass spectrometry analysis, a Slow Off-rate Modified Aptamer (SOMA) scan platform analysis, liquid chromatography (LC) fractionation, Mesoscale platform, electrochemiluminescence detection, or an OLINK Proximity Extension Assay based proteomic platform analysis.

37. The method of claim 2, wherein the kidney function decline is progression from normal kidney function to chronic kidney disease, or from chronic kidney disease to end stage kidney disease (ESKD).

38. (canceled)

39. The method of claim 2, wherein the human subject has type 1 diabetes (T1D), type 2 diabetes (T2D), diabetic kidney disease, cystinosis, glomerulonephritis, polycystic kidney disease, IgA nephropathy, early progressive renal decline, or late progressive renal decline.

40-44. (canceled)
